SEQUENCE LISTING (25 sequences)

SEQ ID NO: 1 - DNA, 1662 bp, Homo sapiens
  source 1..1662: mol_type = genomic DNA
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatcctccagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtg

SEQ ID NO: 2 - DNA, 1314 bp, Homo sapiens
  source 1..1314: mol_type = genomic DNA
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggccatgagaggcagtccgagggatcctccagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtg

SEQ ID NO: 3 - DNA, 619 bp, Homo sapiens
  source 1..619: mol_type = genomic DNA
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaaggccatgagaggcagtccgagggatcctccagcccgcagctccaggagtctgtcttccacctgctggtgcccagtg

SEQ ID NO: 4 - DNA, 1662 bp, Homo sapiens; identical to SEQ ID NO: 1
  source 1..1662: mol_type = genomic DNA

SEQ ID NO: 5 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 6 - protein, 522 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 7 - protein, 438 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGHERQSEGSSSPQLQESVFHLLVPSVILVLLAVGGLLFYRWRRRSHQEPQRADSPLEQPEGSPLTQDDRQVELPV

SEQ ID NO: 8 - protein, 406 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGHERQSEGSSSPQLQESVFHLLVPSVILVLLAVGGLLFYRWRRRSHQEPQRADSPLEQPEGSPLTQDDRQVELPV

SEQ ID NO: 9 - protein, 256 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQGHERQSEGSSSPQLQESVFHLLVPSVILVLLAVGGLLFYRWRRRSHQEPQRADSPLEQPEGSPLTQDDRQVELPV

SEQ ID NO: 10 - protein, 224 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQGHERQSEGSSSPQLQESVFHLLVPSVILVLLAVGGLLFYRWRRRSHQEPQRADSPLEQPEGSPLTQDDRQVELPV

SEQ ID NO: 11 - protein, 245 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKGKAKNPPGDSSLHWAAMALPALFSLIIGFAFGALYWKKRQPSLTRAVENIQINEEDNEISMLQEKEREFQEV

SEQ ID NO: 12 - DNA, 738 bp, Homo sapiens
  source 1..738: mol_type = genomic DNA
atgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaa

SEQ ID NO: 13 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 14 - DNA, 822 bp, Homo sapiens
  source 1..822: mol_type = genomic DNA
atgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgcagccagctcccttaggaatgacagcagtagcagtaataggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaa

SEQ ID NO: 15 - DNA, 1405 bp, Homo sapiens
  source 1..1405: mol_type = genomic DNA
ccgcctcgcgccgagactagaagcgctgcgggaagcagggacagtggagagggcgctgcgctcgggctacccaatgcgtggactatctgccgccgctgttcgtgcaatatgctggagctccagaacagctaaacggagtcgccacaccactgtttgtgctggatcgcagcgctgcctttccttatgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgcagccagctcccttaggaatgacagcagtagcagtaataggaaggccaaaaatccccctggagactccagcctacactgggcagccatggcattgccagcattgttttctcttataattggctttgcttttggagccttatactggaagaagagacagccaagtcttacaagggcagttgaaaatatacaaattaatgaagaggataatgagataagtatgttgcaagagaaagagagagagtttcaagaagtgtaaattgtggcttgtatcaacactgttactttcgtacattggctggtaacagttcatgtttgcttcataaatgaagcagctttaaacaaattcatattctgtctggagtgacagaccacatctttatctgttcttgctacccatgactttatatggatgattcagaaattggaacagaatgttttactgtgaaactggcactgaattaatcatctataaagaagaacttgcatggagcaggactctattttaaggactgcgggacttgggtctcatttagaacttgcagctgatgttggaagagaaagcacgtgtctcagactgcatgtaccatttgcatggctccagaaatgtctaaatgctgaaaaaacacctagctttattcttcagatacaaactgcag

SEQ ID NO: 16 - protein, 189 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVA

SEQ ID NO: 17 - DNA, 567 bp, Homo sapiens
  source 1..567: mol_type = genomic DNA
atgaagaagacacaaacttggattctcacttgcatttatcttcagctgctcctatttaatcctctcgtcaaaactgaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgca

SEQ ID NO: 18 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 19 - DNA, 435 bp, Homo sapiens
  source 1..435: mol_type = genomic DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 20 - protein, 127 aa, Homo sapiens [sequence omitted]

SEQ ID NO: 21 - DNA, 352 bp, Homo sapiens
  source 1..352: mol_type = genomic DNA
gccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 22 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 23 - DNA, 459 bp, Homo sapiens
  source 1..459: mol_type = genomic DNA
atgagccgcctgcccgtcctgctcctgctccaactcctggtccgccccggactccaagctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttttga

SEQ ID NO: 24 - protein, 133 aa, Homo sapiens
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 25 - DNA, 402 bp, Homo sapiens
  source 1..402: mol_type = genomic DNA
gctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttttga

(19) United States
(12) Patent Application Publication
(10) Pub. No.: US 2026/0033467 A1
(43) Pub. Date: Feb. 5, 2026

(54) METHODS AND MODELS FOR ASSESSING EFFICACY OF IMMUNOTHERAPIES

(71) Applicants: The Jackson Laboratory, Bar Harbor, ME (US); University of Massachusetts, Westborough, MA (US)

(72) Inventors: Leonard D. Shultz, Bar Harbor, ME (US); James Keck, Bar Harbor, ME (US); Dale L. Greiner, Westborough, MA (US); Michael A. Brehm, Westborough, MA (US)

(73) Assignees: The Jackson Laboratory, Bar Harbor, ME (US); University of Massachusetts, Westborough, MA (US)

(21) Appl. No.: 19/301,265

(22) Filed: Aug. 15, 2025

Related U.S. Application Data
(63) Continuation of application No. 18/758,838, filed on Jun. 28, 2024, now Pat. No. 12,409,237.

(51) Int. Cl. A01K 67/0278; A01K 67/0271; A61K 49/00; C07K 14/53; C07K 14/535; C07K 14/54

(52) U.S. Cl. CPC A01K 67/0278; A01K 67/0271; A61K 49/0008; C07K 14/53; C07K 14/535; C07K 14/5403; A01K 2207/12; A01K 2207/15; A01K 2217/072; A01K 2217/15; A01K 2227/105; A01K 2267/0331

(57) ABSTRACT

A method of identifying anti-tumor activity of a test substance is provided along with a genetically-modified, immunodeficient mouse and methods of use, wherein the genetically-modified, immunodeficient mouse enables in vivo investigation of the interactions between the human immune system and human cancer.

### RELATED APPLICATIONS

This application is a continuation of U.S. application Ser. No. 18/758,838, filed Jun. 28, 2024, which is herein incorporated by reference in its entirety.

### GRANT REFERENCE

This invention was made with government support under Grant Nos. CA034196, ODO18259, DK104218, and AI132963, awarded by the National Institutes of Health. The Government has certain rights in the invention.

### REFERENCE TO AN ELECTRONIC SEQUENCE LISTING

The contents of the electronic sequence listing (J02277044US04-SEQ-HJD.xml; Size: 36,284 bytes; and Date of Creation: Aug. 15, 2025) are herein incorporated by reference in its entirety.

### BACKGROUND OF THE INVENTION

There is a continuing need for new “humanized” mouse models that allow robust engraftment of human hematopoietic stem cells along with human-patient derived tumor xenografts and/or human tumor cell lines to enable in vivo investigation of the interactions between the human immune system and human cancer.

### SUMMARY OF THE INVENTION

A method of identifying anti-tumor activity of a test substance according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; and wherein the genetically-modified, immunodeficient mouse includes a human hematopoietic stem cell; administering a human tumor cell to the genetically-modified, immunodeficient mouse, wherein the human tumor cell forms a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity.

A method of identifying anti-tumor activity of a test substance according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1,wherein the mouse is a genetically modified NOD.Cg-Prkdc^{scid} Il2rg^{tm1Wjl}/SzJ (NSG) mouse; a genetically modified NOD.Cg-Rag1^{tm/Mom} Il2rg^{tm1Wjl}/SzJ (NRG) mouse or a genetically modified NOD.Cg-Prkdc^{scid} Il2rg^{tm1Sug}/JicTac (NOG) mouse; and wherein the genetically-modified, immunodeficient mouse includes a human hematopoietic stem cell; administering a human tumor cell to the genetically-modified, immunodeficient mouse, wherein the human tumor cell forms a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity.

A method of identifying anti-tumor activity of a test substance according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; and wherein the genetically-modified, immunodeficient mouse includes a CD34+ human hematopoietic stem cell; administering a human tumor cell to the genetically-modified, immunodeficient mouse, wherein the human tumor cell forms a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity.

A method of identifying anti-tumor activity of a test substance according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm/Mom} Il2rg^{tm1Wjl}/SzJ (NRG) mouse or a genetically modified NOD.Cg-Prkdc^{scid} Il2rg^{tm1Sug}/JicTac (NOG) mouse; and wherein the genetically-modified, immunodeficient mouse includes a CD34+ human hematopoietic stem cell; administering a human tumor cell to the genetically-modified, immunodeficient mouse, wherein the human tumor cell forms a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity.

A method of identifying anti-tumor activity of a test substance according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; and wherein the genetically-modified, immunodeficient mouse includes a differentiated human hematopoietic stem cell selected from the group consisting of a human myeloid progenitor cell, a human lymphoid progenitor cell, a human CD33⁺ myeloid cell, a human mast cell, a human monocyte, a human macrophage, human myeloid dendritic cell, a human B cell, a human plasma cell, a human T cell, a human T helper cell, a human cytotoxic T cell, a human T_{reg} cell, a human natural killer cell, and any two or more thereof; administering a human tumor cell to the genetically-modified, immunodeficient mouse, wherein the human tumor cell forms a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity.

A method of identifying anti-tumor activity of a test substance according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; and wherein the genetically-modified, immunodeficient mouse includes a differentiated human hematopoietic stem cell selected from the group consisting of a human myeloid progenitor cell, a human lymphoid progenitor cell, a human CD33⁺ myeloid cell, a human mast cell, a human monocyte, a human macrophage, human myeloid dendritic cell, a human B cell, a human plasma cell, a human T cell, a human T helper cell, a human cytotoxic T cell, a human T_{reg} cell, a human natural killer cell, and any two or more thereof; administering a human tumor cell to the genetically-modified, immunodeficient mouse, wherein the human tumor cell forms a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity.

A method of identifying anti-tumor activity of a test substance according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; and wherein the genetically-modified, immunodeficient mouse includes a human leukocyte selected from the group consisting of: CD45⁺ leukocyte, CD20⁺ leukocyte, CD20⁺CD45⁺ leukocyte, CD3⁺ leukocyte, CD3⁺CD45⁺ leukocyte, CD33⁺ leukocyte, CD33⁺CD45⁺ leukocyte, CD14⁺ leukocyte, CD14⁺CD45⁺ leukocyte, CD56⁺ leukocyte, CD56⁺CD45⁺ leukocyte, and any two or more thereof; administering a human tumor cell to the genetically-modified, immunodeficient mouse, wherein the human tumor cell forms a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity.

A method of identifying anti-tumor activity of a test substance according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; and wherein the genetically-modified, immunodeficient mouse includes a human leukocyte selected from the group consisting of: CD45⁺ leukocyte, CD20⁺ leukocyte, CD20⁺CD45⁺ leukocyte, CD3⁺ leukocyte, CD3⁺CD45⁺ leukocyte, CD33⁺ leukocyte, CD33⁺CD45⁺ leukocyte, CD14⁺ leukocyte, CD14⁺CD45⁺ leukocyte, CD56⁺ leukocyte, CD56⁺CD45³⁰ leukocyte, and any two or more thereof; administering a human tumor cell to the genetically-modified, immunodeficient mouse, wherein the human tumor cell forms a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity.

A method of identifying anti-tumor activity of a test substance according to aspects of the present invention optionally includes comparing the response to a standard to determine the effect of the test substance on the xenogeneic tumor cell, wherein an inhibitory effect of the test substance on the xenogeneic tumor cell identifies the test substance as having anti-tumor activity.

The test substance can be an immunotherapeutic agent, such as an immune checkpoint inhibitor. Immune checkpoint inhibitors include, but are not limited to, a PD-1 inhibitor, PD-L1 inhibitor, or CTLA-4 inhibitor. Immune checkpoint inhibitors include, but are not limited to, atezolizumab, avelumab, durvalumab, ipilimumab, nivolumab, or pembrolizumab, or an antigen-binding fragment of any one of the foregoing. The test substance can be an antibody. The test substance can be an anti-cancer agent.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, and wherein the genetically-modified, immunodeficient mouse expresses hSCF, hGM-CSF, hIL-3, and hCSF1.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1 and wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; and wherein the genetically-modified, immunodeficient mouse includes a human hematopoictic stem cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; and wherein the genetically-modified, immunodeficient mouse includes a human hematopoietic stem cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; and wherein the genetically-modified, immunodeficient mouse includes a CD34⁺ human hematopoietic stem cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; and wherein the genetically-modified, immunodeficient mouse includes a CD34⁺ human hematopoietic stem cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; and wherein the genetically-modified, immunodeficient mouse includes a differentiated human hematopoietic stem cell selected from the group consisting of a human myeloid progenitor cell, a human lymphoid progenitor cell, a human CD33⁺ myeloid cell, a human mast cell, a human monocyte, a human macrophage, human myeloid dendritic cell, a human B cell, a human plasma cell, a human T cell, a human T helper cell, a human cytotoxic T cell, a human Treg cell, a human natural killer cell, and any two or more thereof.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; and wherein the genetically-modified, immunodeficient mouse includes a differentiated human hematopoietic stem cell selected from the group consisting of a human myeloid progenitor cell, a human lymphoid progenitor cell, a human CD33⁺ myeloid cell, a human mast cell, a human monocyte, a human macrophage, human myeloid dendritic cell, a human B cell, a human plasma cell, a human T cell, a human T helper cell, a human cytotoxic T cell, a human Treg cell, a human natural killer cell, and any two or more thereof.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; and wherein the genetically-modified, immunodeficient mouse includes a human leukocyte selected from the group consisting of: CD45⁺ leukocyte, CD20⁺ leukocyte, CD20⁺CD45⁺ leukocyte, CD3⁺ leukocyte, CD3⁺CD45⁺ leukocyte, CD33⁺ leukocyte, CD33⁺CD45⁺ leukocyte, CD14⁺ leukocyte, CD14⁺CD45⁺ leukocyte, CD56⁺ leukocyte, CD56⁺CD45⁺ leukocyte, and any two or more thereof.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2r^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; and wherein the genetically-modified, immunodeficient mouse includes a human leukocyte selected from the group consisting of: CD45⁺ leukocyte, CD20⁺ leukocyte, CD20⁺CD45⁺ leukocyte, CD3⁺ leukocyte, CD3⁺CD45⁺ leukocyte, CD33⁺ leukocyte, CD33⁺CD45⁺ leukocyte, CD14⁺ leukocyte, CD14⁺CD45⁺ leukocyte, CD56⁺ leukocyte, CD56⁺CD45⁺ leukocyte, and any two or more thereof.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, and further comprises a human xenograft comprising a human tumor cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1 wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse, and further comprises a human xenograft comprising a human tumor cell.

A genetically-modified, immunodeficient mouse is provided according to aspects

of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; wherein the genetically-modified, immunodeficient mouse includes a human hematopoietic stem cell, and further comprises a human xenograft comprising a human tumor cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; wherein the genetically-modified, immunodeficient mouse includes a human hematopoietic stem cell, and further comprises a human xenograft comprising a human tumor cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; wherein the genetically-modified, immunodeficient mouse includes a CD34+ human hematopoietic stem cell, and further comprises a human xenograft comprising a human tumor cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; wherein the genetically-modified, immunodeficient mouse includes a CD34⁺ human hematopoietic stem cell, and further comprises a human xenograft comprising a human tumor cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1; wherein the genetically-modified, immunodeficient mouse includes a differentiated human hematopoietic stem cell selected from the group consisting of: a human myeloid progenitor cell, a human lymphoid progenitor cell, a human CD33⁺ myeloid cell, a human mast cell, a human monocyte, a human macrophage, human myeloid dendritic cell, a human B cell, a human plasma cell, a human T cell, a human T helper cell, a human cytotoxic T cell, a human T_{reg} cell, a human natural killer cell, and any two or more thereof, and further comprises a human xenograft comprising a human tumor cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wil}/SzJ (NRG) mouse or a genetically modified NOD.Cg-Prkdc^{scid} Il2rg^{tm1Sug}/JicTac (NOG) mouse; wherein the genetically-modified, immunodeficient mouse includes a differentiated human hematopoietic stem cell selected from the group consisting of: a human myeloid progenitor cell, a human lymphoid progenitor cell, a human CD33⁺ myeloid cell, a human mast cell, a human monocyte, a human macrophage, human myeloid dendritic cell, a human B cell, a human plasma cell, a human T cell, a human T helper cell, a human cytotoxic T cell, a human Treg cell, a human natural killer cell, and any two or more thereof, and further comprises a human xenograft comprising a human tumor cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1;

wherein the genetically-modified, immunodeficient mouse includes a human leukocyte selected from the group consisting of: CD45⁺ leukocyte, CD20⁺ leukocyte, CD20⁺CD45⁺ leukocyte, CD3⁺ leukocyte, CD3⁺CD45⁺ leukocyte, CD33⁺ leukocyte, CD33⁺CD45⁺ leukocyte, CD14⁺ leukocyte, CD14⁺CD45⁺ leukocyte, CD56⁺ leukocyte, CD56⁺CD45⁺ leukocyte, and any two or more thereof, and further comprises a human xenograft comprising a human tumor cell.

A genetically-modified, immunodeficient mouse is provided according to aspects of the present invention, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sus}/JicTac (NOG) mouse; wherein the genetically-modified, immunodeficient mouse includes a human leukocyte selected from the group consisting of: CD45⁺ leukocyte, CD20⁺ leukocyte, CD20⁺CD45⁺ leukocyte, CD3⁺ leukocyte, CD3⁺CD45⁺ leukocyte, CD33⁺ leukocyte, CD33⁺CD45⁺ leukocyte, CD14⁺ leukocyte, CD14⁺CD45⁺ leukocyte, CD56⁺ leukocyte, CD56⁺CD45⁺ leukocyte, and any two or more thereof, and further comprises a human xenograft comprising a human tumor cell.

According to aspects of the present invention, a genetically-modified, immunodeficient mouse is provided wherein any one or more of the nucleotide sequences encoding hSCF, hGM-CSF, hIL-3, or hCSF1 is operably-linked to a constitutive promoter.

According to aspects of the present invention, a genetically-modified, immunodeficient mouse is provided wherein said mouse comprises, in the absence of an immunological challenge, one, two, three or all of: at least about 20% of the human CD45⁺ leukocytes of the mouse are CD3⁺CD45⁺ leukocytes; at least about 10% of the human CD45⁺ leukocytes of the mouse are CD33⁺CD45⁺ leukocytes; at least about 5% of the human CD45⁺ leukocytes of the mouse are CD14⁺CD45⁺ leukocytes; at least about 0.5% of the human CD45⁺ leukocytes of the mouse are CD56⁺CD45⁺leukocytes.

According to aspects of the present invention, a genetically-modified, immunodeficient mouse is provided wherein the mouse expresses one or more of: a human cytokine selected from the group consisting of human interleukin-8, human interleukin-1β, human tumor-necrosis factor, human interleukin-12p70, and human interleukin-6.

A method of making a genetically-modified, immunodeficient humanized mouse model according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, and wherein the genetically-modified, immunodeficient mouse expresses hSCF, hGM-CSF, hIL-3, and hCSF1; and administering human hematopoietic stem cells to the genetically-modified, immunodeficient mouse.

A method of making a genetically-modified, immunodeficient humanized mouse model according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1 and wherein the mouse is a genetically modified NOD.Cg-Prkdc^{scid} Il2r^{tm1Wjl}/SzJ (NSG) mouse; a genetically modified NOD.Cg-Rag1^{tm1Mom} Il2r^{tm1Wjl}/SzJ (NRG) mouse or a genetically modified NOD.Cg-Prkdc^{scid} Il2rg^{tm1Sus}/JicTac (NOG) mouse; and administering human hematopoietic stem cells to the genetically-modified, immunodeficient mouse.

A method of making a genetically-modified, immunodeficient humanized mouse model according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, and wherein the genetically-modified, immunodeficient mouse expresses hSCF, hGM-CSF, hIL-3, and hCSF1; administering human hematopoietic stem cells to the genetically-modified, immunodeficient mouse; and administering a human xenograft comprising a human tumor cell to the genetically-modified, immunodeficient mouse.

A method of making a genetically-modified, immunodeficient humanized mouse model according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1 and wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wil}/SzJ (NRG) mouse or a genetically modified NOD.Cg-Prkdc^{scid} Il2rg^{tm1Sug}/JicTac (NOG) mouse; administering human hematopoietic stem cells to the genetically-modified, immunodeficient mouse; and administering a human xenograft comprising a human tumor cell to the genetically-modified, immunodeficient mouse.

A method of making a genetically-modified, immunodeficient humanized mouse model according to aspects of the present invention optionally includes conditioning the genetically-modified, immunodeficient mouse to reduce mouse hematopoietic cells of the mouse prior to administering human hematopoietic stem cells. For example, a method of making a genetically-modified, immunodeficient humanized mouse model according to aspects of the present invention optionally includes irradiating the genetically-modified, immunodeficient mouse and/or administering a radiomimetic drug to the genetically-modified, immunodeficient mouse.

A method of making a genetically-modified, immunodeficient humanized mouse model according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, and wherein the genetically-modified, immunodeficient mouse expresses hSCF, hGM-CSF, hIL-3, and hCSF1; administering human hematopoietic stem cells to the genetically-modified, immunodeficient mouse; and administering a human xenograft comprising a human tumor cell to the genetically-modified, immunodeficient mouse, wherein the human hematopoietic stem cell and the human tumor cell comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 matching HLA alleles.

A method of making a genetically-modified, immunodeficient humanized mouse model according to aspects of the present invention includes providing a genetically-modified, immunodeficient mouse, wherein the genetically-modified, immunodeficient mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, wherein the genetically-modified, immunodeficient mouse expresses hSCF, hGM-CSF, hIL-3, and hCSF1 and wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sug}/JicTac (NOG) mouse; administering human hematopoietic stem cells to the genetically-modified, immunodeficient mouse; and administering a human xenograft comprising a human tumor cell to the genetically-modified, immunodeficient mouse, wherein the human hematopoietic stem cell and the human tumor cell comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 matching HLA alleles.

General aspects of the disclosure related to mouse models of interactions between the human immune system and human cancer. According to specific aspects, a genetically-modified, immunodeficient mouse is provided which expresses hSCF, hGM-CSF, hIL-3, and hCSF1, along with methods of use thereof.

### BRIEF DESCRIPTION OF THE DRAWINGS

FIG. 1A is a chart depicting percentages of flow-cytometry-gated blood cells expressing human CD45 as a percentage of total blood cells in samples from NSG mice, NSG-SGM3 mice and NSG-QUAD mice 10-weeks after administration of 100,000 human CD34⁺ hematopoietic stem cells.

FIG. 1B is a chart depicting percentages of flow-cytometry-gated blood cells expressing human CD20 as a percentage of CD45-positive blood cells in samples from NSG mice, NSG-SGM3 mice and NSG-QUAD mice 10-weeks after administration of 100,000 human CD34⁺ hematopoietic stem cells.

FIG. 1C is a chart depicting percentages of flow-cytometry-gated blood cells expressing human CD3 as a percentage of CD45-positive blood cells in samples from NSG mice, NSG-SGM3 mice and NSG-QUAD mice 10-weeks after administration of 100,000 human CD34⁺ hematopoietic stem cells.

FIG. 1D is a chart depicting percentages of flow-cytometry-gated blood cells expressing human CD33 as a percentage of CD45-positive blood cells in samples from NSG mice, NSG-SGM3 mice and NSG-QUAD mice 10-weeks after administration of 100,000 human CD34⁺ hematopoietic stem cells.

FIG. 1E is a chart depicting percentages of flow-cytometry-gated blood cells expressing human CD14 as a percentage of CD45-positive blood cells in samples from NSG mice, NSG-SGM3 mice and NSG-QUAD mice 10-weeks after administration of 100,000 human CD34⁺ hematopoietic stem cells.

FIG. 1F is a chart depicting percentages of flow-cytometry-gated blood cells

expressing human CD56 as a percentage of CD45-positive blood cells in samples from NSG mice, NSG-SGM3 mice and NSG-QUAD mice 10-weeks after administration of 100,000 human CD34⁺ hematopoietic stem cells;

FIG. 2A is a chart depicting the concentration of human IL8 in serum samples from NSG mice, NSG-SGM3/+mice, NSG-SGM3/SGM3, and NSG-QUAD mice which were administered 100,000 human CD34⁺ hematopoietic stem cells followed by 0.15 μg intravenous lipopolysaccharide 10-weeks later. The serum samples were obtained 6 hours after administration of the lipopolysaccharide and the human IL8 was measured by bead-based immunoassay.

FIG. 2B is a chart depicting the concentration of human IL1-B in serum samples from NSG mice, NSG-SGM3/+ mice, NSG-SGM3/SGM3, and NSG-QUAD mice which were administered 100,000 human CD34⁺ hematopoietic stem cells followed by 0.15 μg intravenous lipopolysaccharide 10-weeks later. The serum samples were obtained 6 hours after administration of the lipopolysaccharide and the human IL1-B was measured by bead-based immunoassay.

FIG. 2C is a chart depicting the concentration of human TNF in serum samples from NSG mice, NSG-SGM3/+ mice, NSG-SGM3/SGM3, and NSG-QUAD mice which were administered 100,000 human CD34⁺ hematopoietic stem cells followed by 0.15 μg intravenous lipopolysaccharide 10-weeks later. The serum samples were obtained 6 hours after administration of the lipopolysaccharide and the human TNF was measured by bead-based immunoassay.

FIG. 2D is a chart depicting the concentration of human IL12p70 in serum samples from NSG mice, NSG-SGM3/+ mice, NSG-SGM3/SGM3, and NSG-QUAD mice which were administered 100,000 human CD34⁺ hematopoietic stem cells followed by 0.15 μg intravenous lipopolysaccharide 10-weeks later. The serum samples were obtained 6 hours after administration of the lipopolysaccharide and the human IL12p70 was measured by bead-based immunoassay.

FIG. 2E is a chart depicting the concentration of human IL6 in serum samples

from NSG mice, NSG-SGM3/+ mice, NSG-SGM3/SGM3, and NSG-QUAD mice which were administered 100,000 human CD34⁺ hematopoietic stem cells followed by 0.15 μg intravenous lipopolysaccharide 10-weeks later. The serum samples were obtained 6hours after administration of the lipopolysaccharide and the human IL6 was measured by bead-based immunoassay.

FIG. 3A is a graph depicting the mean tumor volume of tumors formed by a patient-derived xenograft (PDX) of human BR1126 breast cancer cells in NSG-QUAD mice (also called hNSG-Quad mice) compared to the mean tumor volume of tumors formed by a PDX of human BR1126 breast cancer cells NSG-SGM3 mice (also called hNSG-SGM3 mice).

FIG. 3B is a graph depicting the mean tumor volume of tumors formed by a PDX of human MDA-MB231 breast cancer cells in NSG-QUAD mice compared to the mean tumor volume of tumors formed by a PDX of human MDA-MB231 breast cancer cells NSG-SGM3 mice.

FIG. 3C is a graph depicting the mean tumor volume of tumors formed by a PDX

of human TM1149 breast cancer cells in NSG-QUAD mice compared to the mean tumor volume of tumors formed by a PDX of human TM1149 breast cancer cells in NSG-SGM3 mice.

FIG. 3D is a graph depicting the mean tumor volume of tumors formed by a PDX of human BL0293 bladder cancer cells in NSG-QUAD mice compared to the mean tumor volume of tumors formed by a PDX of human BL0293 bladder cancer cells in NSG-SGM3 mice.

FIG. 4A is a chart showing characteristics of blood cells obtained from NSG-SGM3 and NSG-Quad mice to which 100,000 human CD34⁺ hematopoietic stem cells were administered followed by a subcutaneous TM1149 xenograft 7-12 weeks later. PDX tumors harvested 17 weeks after xenograft administration were analyzed by flow cytometry and the percent of human CD45-positive (hCD45⁺) in the population of total live cells in the sample is shown.

FIG. 4B is a chart showing characteristics of blood cells obtained from NSG-SGM3 and NSG-Quad mice to which 100,000 human CD34⁺ hematopoietic stem cells were administered followed by a subcutaneous TM1149 xenograft 7-12 weeks later. PDX tumors harvested 17 weeks after xenograft administration were analyzed by flow cytometry and the percent of human CD3-positive (hCD3⁺) in the population of CD45⁺ cells in the sample is shown.

FIG. 4C is a chart showing characteristics of blood cells obtained from NSG-SGM3 and NSG-Quad mice to which 100,000 human CD34⁺ hematopoietic stem cells were administered followed by a subcutaneous TM1149 xenograft 7-12 weeks later. PDX tumors harvested 17 weeks after xenograft administration were analyzed by flow cytometry and the percent of human CD20-positive (hCD20⁺) in the population of CD45⁺ cells in the sample is shown.

FIG. 4D is a chart showing characteristics of blood cells obtained from NSG-SGM3 and NSG-Quad mice to which 100,000 human CD34⁺ hematopoietic stem cells were administered followed by a subcutaneous TM1149 xenograft 7-12 weeks later. PDX tumors harvested 17 weeks after xenograft administration were analyzed by flow cytometry and the percent of human CD33-positive (hCD33⁺) in the population of CD45⁺ cells in the sample is shown.

FIG. 4E is a chart showing characteristics of blood cells obtained from NSG-SGM3 and NSG-Quad mice to which 100,000 human CD34⁺ hematopoietic stem cells were administered followed by a subcutaneous TM1149 xenograft 7-12 weeks later. PDX tumors harvested 17 weeks after xenograft administration were analyzed by flow cytometry and the percent of human CD14-positive (hCD14⁺) in the population of CD45⁺ cells in the sample is shown.

FIG. 4F is a chart showing characteristics of blood cells obtained from NSG-SGM3 and NSG-Quad mice to which 100,000 human CD34⁺ hematopoietic stem cells were administered followed by a subcutaneous TM1149 xenograft 7-12 weeks later. PDX tumors harvested 17 weeks after xenograft administration were analyzed by flow cytometry and the percent of human CD56-positive (hCD56⁺) in the population of CD45⁺ cells in the sample is shown.

FIG. 5 is a graph comparing tumor volumes for NSG-Quad mice (Hu-NSG-Quad mice) that have human CD34⁺ hematopoietic stem cell xenografts and BL0293 patient-derived xenografts and received injections with either pembrolizumab (Keytruda®) or vehicle.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F.M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10:0879695919; and Kursad Turksen (Ed.), Embryonic stem cells: methods and protocols in Methods Mol Biol. 2002;185, Humana Press; Current Protocols in Stem Cell Biology, ISBN: 9780470151808.

The singular terms “a,” “an,” and “the” are not intended to be limiting and include plural referents unless explicitly stated otherwise or the context clearly indicates otherwise.

The term “comprising” refers to an open group, e.g., a group comprising members A, B, and C can also include additional members. The term “consisting of” refers to a closed group, e.g., a group consisting of members A, B, and C does not include any additional members.

The term “xenogeneic” is used herein with reference to a host cell or organism to indicate that the material referred to as “xenogeneic” is derived from another species than that of the host cell or organism.

A genetically-modified, immunodeficient mouse which expresses human stem cell factor (hSCF), human granulocyte-macrophage colony-stimulating factor (hGM-CSF), human interleukin-3 (hIL-3), and human colony-stimulating factor 1 (hCSF1) (called an “immunodeficient QUAD mouse” herein) is provided according to aspects of the present invention.

Various aspects of the invention relate to an immunodeficient QUAD mouse including nucleotide sequences encoding hSCF, hGM-CSF, hIL-3, and hCSF1 in its genome wherein the immunodeficient QUAD mouse expresses hSCF, hGM-CSF, hIL-3, and hCSF1 proteins. Nucleic acid sequences are shown and described herein which encode hSCF, hGM-CSF, hIL-3, hCSF1 proteins and variants thereof. It will be appreciated by those of ordinary skill in the art that, due to the degenerate nature of the genetic code, alternate nucleic acid sequences encode hCSF1, hGM-SCF, hSCF, hIL-3 and variants thereof and that such alternate nucleic acids may be used in compositions and methods described herein.

The terms “human colony-stimulating factor 1,” “human CSF1,” and “hCSF1” are used synonymously herein to refer to a cytokine involved in the proliferation, differentiation, and survival of monocytes, macrophages, and bone marrow progenitor cells. The human CSF1 gene locus resides on chromosome 1, and it occurs as nucleotides 109,910,242-109,930,992 of the Genome Reference Consortium Human Reference 38 genome (GRCh38/hg38). The human CSF1 gene is expressed as at least four transcript variants (see, e.g., NCBI Reference Sequences NM_000757.5, NM_172210.2, NM_172211.3, and NM_172212.2), which produce at least three protein isoforms, a, b, and c (sec, e.g., NCBI Reference Sequences NP_000748.3, NP_757349.1, NP_757350.1, and NP_757351.1). The term “hCSF1” includes variants of hCSF1 amino acid sequences specifically identified herein. Human CSF1 proteins are identified herein as SEQ ID NO:5, SEQ ID NO:6, SEQ ID NO:7, SEQ ID NO:8, SEQ ID NO:9, SEQ ID NO: 10, and variants thereof are useful in the present invention.

An immunodeficient QUAD mouse according to aspects of the present invention includes in its genome a nucleic acid encoding hCSF1 operably linked to a promoter wherein the hCSF1 includes the amino acid sequence of SEQ ID NO:5, SEQ ID NO:6, SEQ ID NO:7, SEQ ID NO:8, SEQ ID NO:9, SEQ ID NO: 10, the amino acid sequence encoded by the complement of a nucleic acid which hybridizes to SEQ ID NO:1 under high stringency hybridization conditions, the amino acid sequence encoded by the complement of a nucleic acid which hybridizes to SEQ ID NO:2 under high stringency hybridization conditions, the amino acid sequence encoded by the complement of a nucleic acid which hybridizes to SEQ ID NO:3 under high stringency hybridization conditions, or the amino acid sequence encoded by the complement of a nucleic acid which hybridizes to SEQ ID NO:4 under high stringency hybridization conditions.

The terms “human stem cell factor,” “human SCF,” and “hSCF” are used synonymously herein to refer to a well-known cytokine that binds to the c-Kit receptor (CD117). SCF is also known as kit ligand, SF, Kitl, KL-1 and other names. Various isoforms of SCF are known including transmembrane and soluble isoforms generated by alternative splicing. Particular isoforms include human membrane-associated stem cell factor 248 (SCF²⁴⁸), human membrane-associated stem cell factor 220 (SCF²²⁰) and soluble stem cell factor (SCF), see Anderson, D. M. et al., 1990, Cell 63, 235; Flannagan, J. G. et al., 1991, Cell 64, 1025; Anderson, D. M. et al., 1991, Cell Growth Differ. 2, 373; Martin, F. H. et al., Cell, 63:203, 1990; Huang E. J. et al., Mol. Biol. Cell, 3:349, 1992; and Huang E. et al., Cell, 63:225, 1990. Amino acid sequences of human soluble SCF, human SCF²²⁰ and human SCF²⁴⁸ along with exemplary nucleic acid sequences encoding human soluble SCF, human SCF²²⁰ or human SCF²⁴⁸ are shown herein. The term “hSCF” includes variants of hSCF amino acid sequences specifically identified herein. Human SCF proteins are identified herein as SEQ ID NO:11, SEQ ID NO:13, and SEQ ID NO: 16, and variants thereof are useful in the present invention.

An immunodeficient QUAD mouse according to aspects of the present invention includes in its genome a nucleic acid encoding hSCF operably linked to a promoter wherein the hSCF includes the amino acid sequence of SEQ ID NO:11, SEQ ID NO:13, SEQ ID NO:16, the amino acid sequence encoded by the complement of a nucleic acid which hybridizes to SEQ ID NO:12 under high stringency hybridization conditions, the amino acid sequence encoded by the complement of a nucleic acid which hybridizes to SEQ ID NO:14 under high stringency hybridization conditions, the amino acid sequence encoded by the complement of a nucleic acid which hybridizes to SEQ ID NO: 15 under high stringency hybridization conditions, or the amino acid sequence encoded by the complement of a nucleic acid which hybridizes to SEQ ID NO:17 under high stringency hybridization conditions.

The terms “human granulocyte-macrophage colony-stimulating factor,” “human GM-CSF,” and “hGM-CSF” are used synonymously herein. Human GM-CSF is a well-known cytokine that controls the production, differentiation, and function of granulocytes and macrophages. The term “GM-CSF” includes variants of GM-CSF amino acid sequences specifically identified herein. Human GM-CSF proteins are identified herein as SEQ ID NO:18, and SEQ ID NO:20 and variants thereof in the present invention.

An immunodeficient QUAD mouse according to aspects of the present invention includes in its genome a nucleic acid encoding hGM-CSF operably linked to a promoter wherein the hGM-CSF includes the amino acid sequence of SEQ ID NO:18, SEQ ID NO: 20, the amino acid sequence encoded by the complement of a nucleic acid which hybridizes to SEQ ID NO:19 under high stringency hybridization conditions, or the amino acid sequence encoded by the complement of a nucleic acid which hybridizes to SEQ ID NO:21 under high stringency hybridization conditions.

The terms “human interleukin-3,” “human IL-3,” and “hIL-3” are used synonymously herein. IL-3 is a well-known cytokine that regulates blood-cell production by controlling the production, differentiation and function of granulocytes and macrophages. The term “hIL-3” includes variants of hIL-3 amino acid sequences specifically identified herein. Human IL-3 proteins are identified herein as SEQ ID NO: 22, and SEQ ID NO: 24 and variants thereof in the present invention.

An immunodeficient QUAD mouse according to aspects of the present invention includes in its genome a nucleic acid encoding hIL-3 operably linked to a promoter wherein the hIL-3 includes the amino acid sequence of SEQ ID NO:22, SEQ ID NO:24, the amino acid sequence encoded by the complement of a nucleic acid which hybridizes to SEQ ID NO:23 under high stringency hybridization conditions, or the amino acid sequence encoded by the complement of a nucleic acid which hybridizes to SEQ ID NO: 25 under high stringency hybridization conditions.

Aspects of the present invention relate to an immunodeficient QUAD mouse whose genome includes a nucleotide sequence encoding hCSF1, a nucleotide sequence encoding hSCF, a nucleotide sequence encoding hGM-CSF, and a nucleotide sequence encoding hIL-3, wherein the immunodeficient QUAD mouse expresses hCSF1, hSCF, hGM-CSF, and hIL-3.

According to aspects of the present invention, the included nucleotide sequence encoding hCSF1 has a nucleotide sequence that has at least about 85%, 90%, 91%, 92%, 93%, 94%, 95%, 96%, 97%, 98%, 99%, 99.5%, 99.7%, 99.8% or greater sequence identity with the nucleotide sequence set forth in SEQ ID NO:1, SEQ ID NO:2, SEQ ID NO: 3, or SEQ ID NO:4 and encodes hCSF1 having an amino acid sequence that has at least about 85%, 90%, 91%, 92%, 93%, 94%, 95%, 96%, 97%, 98%, 99%, 99.5%, 99.7%, 99.8% or greater sequence identity with the amino acid sequence set forth in SEQ ID NO: 5, SEQ ID NO:6, SEQ ID NO:7, SEQ ID NO:8, SEQ ID NO:9, or SEQ ID NO: 10.

According to aspects of the present invention, the included nucleotide sequence encoding hCSF1 encodes an mRNA transcript having at least about 85%, 90%, 91%, 92%, 93%, 94%, 95%, 96%, 97%, 98%, 99%, 99.5%, 99.7%, 99.8% or greater sequence identity with the nucleotide sequence set forth in SEQ ID NO:1, SEQ ID NO:2, SEQ ID NO: 3, or SEQ ID NO:4 wherein the deoxythymidine bases of the foregoing sequences are substituted with uridine and wherein the mRNA encodes hCSF1 having an amino acid sequence that has at least about 85%, 90%, 91%, 92%, 93%, 94%, 95%, 96%, 97%, 98%, 99%, 99.5%, 99.7%, 99.8% or greater sequence identity with the amino acid sequence set forth in SEQ ID NO:5, SEQ ID NO:6, SEQ ID NO:7, SEQ ID NO:8, SEQ ID NO: 9, or SEQ ID NO:10.

According to aspects of the present invention, the included nucleotide sequence encoding hSCF has a nucleotide sequence that has at least about 85%, 90%, 91%, 92%, 93%, 94%, 95%, 96%, 97%, 98%, 99%, 99.5%, 99.7%, 99.8% or greater sequence identity with the nucleotide sequence set forth in SEQ ID NO:12, SEQ ID NO:14, SEQ ID NO: 15, or SEQ ID NO:17 and encodes hSCF having an amino acid sequence that has at least about 85%, 90%, 91%, 92%, 93%, 94%, 95%, 96%, 97%, 98%, 99%, 99.5%, 99.7%, 99.8% or greater sequence identity with the amino acid sequence set forth in SEQ ID NO: 11, SEQ ID NO:13, or SEQ ID NO:16.

According to aspects of the present invention, the included nucleotide sequence encoding hSCF encodes an mRNA transcript having at least about 85%, 90%, 91%, 92%, 93%, 94%, 95%, 96%, 97%, 98%, 99%, 99.5%, 99.7%, 99.8% or greater sequence identity with the nucleotide sequence set forth in SEQ ID NO:12, SEQ ID NO: 14, SEQ ID NO: 15, or SEQ ID NO:17 wherein the deoxythymidine bases of the foregoing sequences are substituted with uridine and wherein the mRNA encodes hSCF having an amino acid sequence that has at least about 85%, 90%, 91%, 92%, 93%, 94%, 95%, 96%, 97%, 98%, 99%, 99.5%, 99.7%, 99.8% or greater sequence identity with the amino acid sequence set forth in SEQ ID NO:11, SEQ ID NO:13, or SEQ ID NO:16.

According to aspects of the present invention, the included nucleotide sequence encoding hGM-CSF has a nucleotide sequence that has at least about 85%, 90%, 91%, 92%, 93%, 94%, 95%, 96%, 97%, 98%, 99%, 99.5%, 99.7%, 99.8% or greater sequence identity with the nucleotide sequence set forth in SEQ ID NO:19, or SEQ ID NO:21 and encodes hGM-CSF having an amino acid sequence that has at least about 85%, 90%, 91%, 92%, 93%, 94%, 95%, 96%, 97%, 98%, 99%, 99.5%, 99.7%, 99.8% or greater sequence identity with the amino acid sequence set forth in SEQ ID NO:18, or SEQ ID NO: 20.

According to aspects of the present invention, the included nucleotide sequence encoding hGM-CSF encodes an mRNA transcript having at least about 85%, 90%, 91%, 92%, 93%, 94%, 95%, 96%, 97%, 98%, 99%, 99.5%, 99.7%, 99.8% or greater sequence identity with the nucleotide sequence set forth in SEQ ID NO:19, or SEQ ID NO:21 wherein the deoxythymidine bases of the foregoing sequences are substituted with uridine and wherein the mRNA encodes hGM-CSF having an amino acid sequence that has at least about 85%, 90%, 91%, 92%, 93%, 94%, 95%, 96%, 97%, 98%, 99%, 99.5%, 99.7%, 99.8% or greater sequence identity with the amino acid sequence set forth in SEQ ID NO: 18, or SEQ ID NO:20.

According to aspects of the present invention, the included nucleotide sequence encoding hIL-3 has a nucleotide sequence that has at least about 85%, 90%, 91%, 92%, 93%, 94%, 95%, 96%, 97%, 98%, 99%, 99.5%, 99.7%, 99.8% or greater sequence identity with the nucleotide sequence set forth in SEQ ID NO:23, or SEQ ID NO:25 and encodes hIL-3 having an amino acid sequence that has at least about 85%, 90%, 91%, 92%, 93%, 94%, 95%, 96%, 97%, 98%, 99%, 99.5%, 99.7%, 99.8% or greater sequence identity with the amino acid sequence set forth in SEQ ID NO:22, or SEQ ID NO:24.

According to aspects of the present invention, the included nucleotide sequence encoding hIL-3 encodes an mRNA transcript having at least about 85%, 90%, 91%, 92%, 93%, 94%, 95%, 96%, 97%, 98%, 99%, 99.5%, 99.7%, 99.8% or greater sequence identity with the nucleotide sequence set forth in SEQ ID NO:23, or SEQ ID NO:25 wherein the deoxythymidine bases of the foregoing sequences are substituted with uridine and wherein the mRNA encodes hIL-3 having an amino acid sequence that has at least about 85%, 90%, 91%, 92%, 93%, 94%, 95%, 96%, 97%, 98%, 99%, 99.5%, 99.7%, 99.8% or greater sequence identity with the amino acid sequence set forth in SEQ ID NO: 22, or SEQ ID NO:24.

To determine the percent identity of two amino acid sequences or two nucleotide sequences, the sequences are aligned for optimal comparison purposes (e.g., gaps can be introduced in a first amino acid or nucleotide sequence for optimal alignment with a second amino acid or nucleotide sequence using the default parameters of an alignment software program). The amino acids or nucleotides at corresponding amino acid positions or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical aligned positions=total number of aligned positions×100%). In some embodiments, the two sequences have the same length.

The determination of percent identity between two sequences can be accomplished using a mathematical algorithm. A non-limiting example of a mathematical algorithm utilized for the comparison of two sequences is the algorithm of Karlin and Altschul, 1990, PNAS USA 87:2264-68, modified as in Karlin and Altschul, 1993, PNAS USA 90:5873-77. Such an algorithm is incorporated into the NBLAST and

XBLAST programs of Altschul et al, 1990, J. Mol. Biol. 215:403. BLAST nucleotide searches can optionally be performed with the NBLAST nucleotide program parameter set, e.g., for score=100, wordlength=12 to obtain nucleotide sequences homologous to a nucleic acid molecules of the present invention. BLAST protein searches can optionally be performed with the XBLAST program parameter set, e.g., to score 50, wordlength=3 to obtain amino acid sequences homologous to a protein molecule of the present invention. To obtain gapped alignments for comparison purposes, Gapped BLAST can be utilized as described in Altschul et al, 1997, Nucleic Acids Res. 25:3389-02. Alternatively, PSI BLAST can be used to perform an iterated search that detects distant relationships between molecules (id.). When utilizing BLAST, Gapped BLAST, and PSI Blast, the default parameters of the respective programs (e.g., of XBLAST and NBLAST) are used (see, e.g., the NCBI website). Another preferred, non-limiting example of a mathematical algorithm utilized to compare sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used. The Clustal suite of software programs provides an additional method for aligning sequences to determine percent sequence identity.

The percent identity between two sequences is determined using techniques similar to those described above with or without allowing gaps. In calculating percent identity, only exact matches are typically counted.

The terms “hybridization” and “hybridizes” refer to pairing and binding of

complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

Mutations can be introduced using standard molecular biology techniques, such as site-directed mutagenesis and PCR-mediated mutagenesis. One of skill in the art will recognize that one or more amino acid mutations can be introduced without altering the functional properties of the hSCF, hGM-CSF, hIL-3, or hCSF1 proteins.

Conservative amino acid substitutions can be made in the hSCF, hGM-CSF, hIL-3, or hCSF1 proteins to produce hSCF, hGM-CSF, hIL-3, or hCSF1 protein variants. Conservative amino acid substitutions are art recognized substitutions of one amino acid for another amino acid having similar characteristics. For example, each amino acid can be described as having one or more of the following characteristics: electropositive, electronegative, aliphatic, aromatic, polar, hydrophobic, and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine, and valine; aromatic amino acids include phenylalanine, tyrosine, and tryptophan; polar amino acids include aspartate, glutamate, histidine, lysine, asparagine, glutamine, arginine, serine, threonine, and tyrosine; and hydrophobic amino acids include alanine, cysteine, phenylalanine, glycine, isoleucine, leucine, methionine, proline, valine, and tryptophan; and conservative substitutions include substitutions among amino acids within each group. Amino acids can also be described in terms of sterics or relative size, alanine, cysteine, aspartate, glycine, asparagine, proline, threonine, serine, and valine are all typically considered to be small.

The term “nucleic acid” refers to RNA or DNA molecules having more than one nucleotide in any form including single-stranded, double-stranded, oligonucleotide, or polynucleotide. The term “nucleotide sequence” refers to the ordering of nucleotides in a nucleic acid.

Nucleic acids encoding hSCF, hGM-CSF, hIL-3, hCSF1, and variants thereof can be isolated or generated recombinantly or synthetically using well-known methodology.

A nucleotide sequence encoding hSCF, hGM-CSF, hIL-3, or hCSF1 can be operably-linked to a promoter. The promoter can be a constitutive promoter. The promoter can be capable of driving gene expression in a host mouse (e.g., an immunodeficient mouse). For example, the promoter can be the CAG promoter. The CAG promoter includes the cytomegalovirus early enhancer element (“C”), the first exon and the first intron of the chicken beta-actin gene (“A”), and the splice acceptor of the rabbit beta-globulin gene (“G”). The CAG promoter is well known and displays robust expression in mouse cells (see, e.g., Jun-ichi et al, 1989, Gene, 79 (2): 269). The CAG promoter can be modified, for example, to remove exons. Other promoters are known to drive robust gene expression in mice including the cytomegalovirus (CMV) immediate-early promoter and the simian virus 40 (SV40) early promoter. Methods for designing genes and positioning promoters in expression constructs are known (see, e.g., Haruyama et al, 2009, Curr. Protoc. Cell Biol., 19.10).

An immunodeficient QUAD mouse can includes nucleic acids encoding hSCF, hGM-CSF, hIL-3, and hCSF1, and each nucleic acid can be operably-linked to a constitutive promoter.

Immunodeficient mice are provided according to embodiments of the present invention whose genomes include an expression cassette including a nucleotide sequence encoding xenogeneic CSF1, wherein the nucleotide sequence is operably-linked to a promoter and a polyadenylation signal, and the mouse expresses the encoded xenogeneic CSF1.

The terms “expression construct” and “expression cassette” are used herein to refer to a double-stranded recombinant nucleotide sequence containing a desired coding sequence and containing one or more regulatory elements necessary or desirable for the expression of the operably-linked coding sequence. The term “regulatory clement” as used herein refers to a nucleotide sequence that controls some aspect of the expression of nucleotide sequences. Exemplary regulatory elements illustratively include an enhancer, an internal ribosome entry site (IRES), an intron, an origin of replication, a polyadenylation signal (pA), a promoter, a transcription termination sequence, and an upstream regulatory domain, which contribute to the replication, transcription, and post-transcriptional processing of a nucleotide sequence. Those of ordinary skill in the art are capable of selecting and using these and other regulatory elements in an expression construct with no more than routine experimentation. Expression constructs can be generated recombinantly or synthetically using well-known methodology.

The term “operably-linked” as used herein refers to a nucleotide sequence in a functional relationship with a second nucleotide sequence.

A regulatory element included in an expression cassette can be a promoter. The term “promoter” as used herein refers to a regulatory nucleotide sequence operably-linked to a coding nucleotide sequence to be transcribed such as a nucleotide sequence encoding a desired amino acid. A promoter is generally positioned upstream of a nucleotide sequence to be transcribed and provides a site for specific-binding by RNA polymerase and other transcription factors. A promoter can be a constitutive promoter or an inducible promoter. A promoter can optionally provide ubiquitous, tissue-specific, or cell-type specific expression.

Ubiquitous promoters that can be included in an expression construct include, but are not limited to, a 3-phosphoglycerate kinase (PGK-1) promoter, a beta-actin promoter, a ROSA26 promoter, a heat shock protein 70 (Hsp70) promoter, an EF-1 alpha gene encoding elongation factor 1 alpha (EF-1) promoter, an eukaryotic initiation factor 4A (eIF-4A1) promoter, a chloramphenicol acetyltransferase (CAT) promoter, and a cytomegalovirus (CMV) promoter.

Tissue-specific promoters that can be included in an expression construct include, but are not limited to, a promoter of a gene expressed in the hematopoietic system, such as an hSCF promoter, an hCSF1 promoter, a hIL-3 promoter, a hGM-CSF, an IFN-β promoter, a Wiskott-Aldrich syndrome protein (WASP) promoter, a CD45 (also called leukocyte common antigen) promoter, a Flt-1 (fins-like tyrosine kinase, VEGF Receptor 1) promoter, an endoglin (CD105) promoter, and an ICAM-2 (Intracellular Adhesion Molecule 2) promoter.

These and other promoters are known in the art as exemplified in Abboud et al, 2003, J. Histochem & Cytochem., 51 (7): 941-49; Schorpp et al, 1996, Nucl. Acids Res., 24(9): 1787-88; McBurney et al, 1994, Devel. Dynamics, 200:278-93; and Majumder et al, 1996, Blood, 87 (8): 3203-11.

In addition to a promoter, one or more enhancer sequences can be included such as, but not limited to, the cytomegalovirus (CMV) early enhancer element and the SV40 enhancer element.

Additional included sequences include an intron sequence such as the beta globin intron or a generic intron, a transcription termination sequence, and an mRNA polyadenylation (pA) sequence such as, but not limited to SV40-pA, beta-globin-pA, hIL-3-pA, hGM-CSF-pA, hSCF-pA, and hCSF1-pA.

An expression construct can optionally include sequences necessary for amplification in bacterial cells, such as a selection marker (e.g., kanamycin or ampicillin resistance gene) and an origin of replication.

For methods of DNA injection of an expression construct into a mouse preimplantation embryo, the expression construct can be linearized before injection into the embryo. Preferably, the expression construct is injected into fertilized oocytes. Fertilized oocytes are collected from superovulated females the day after mating (0.5 days post coitum) and injected with the expression construct. The injected oocytes are either cultured overnight or transferred directly into oviducts of 0.5-day post coitum pseudopregnant females. Methods for superovulation, harvesting of oocytes, expression construct injection, and embryo transfer are known in the art and described, for example, in Manipulating the Mouse Embryo: A Laboratory Manual, 3^{rd} edition, Cold Spring Harbor Laboratory Press; Dec. 15, 2002, ISBN-10:0879695919. Offspring can be tested for the presence of the expression construct, or relevant portion thereof, by DNA analysis, such as by PCR, Southern blot, or nucleic acid sequencing. Mice that carry the expression construct, or relevant portion thereof, can be tested for protein expression such as by ELISA or Western blot analysis.

The xenogeneic nucleotide sequences encoding hSCF, hGM-CSF, hIL-3, and hCSF1 can be integrated into the genome of some or all of the cells of the mouse. For example, the xenogeneic nucleotide sequences are integrated into the genomes of germline cells of the mouse according to aspects of the present invention thereby enabling the inheritance of the genes to progeny of the mouse.

Alternatively, an expression construct can be transfected into stem cells (embryonic stem cells or induced pluripotent stem cells) using well-known methods, such as electroporation, calcium-phosphate precipitation, or lipofection. The cells are screened for integration of the expression construct, or relevant portion thereof, by DNA analysis, such as PCR, Southern blot, or nucleic acid sequencing. Cells with the correct integration can be tested for functional expression by protein analysis for hSCF, hGM-CSF, hIL-3, and/or hCSF1 using, for example, ELISA or Western blot analysis.

Embryonic stem cells are grown in media optimized for the particular cell line. Typically, embryonic stem cell media contains 15% fetal bovine serum (FBS) or synthetic or semi-synthetic equivalents, 2 mM glutamine, 1 mM sodium pyruvate, 0.1 mM non-essential amino acids, 50 U/mL penicillin and streptomycin, 0.1 mM 2-mercaptoethanol, and 1000 U/mL LIF (plus, for some cell lines chemical inhibitors of differentiation) in Dulbecco's Modified Eagle Media (DMEM). A detailed description is known in the art (Tremml et al, 2008, Current Protocols in Stem Cell Biology, Chapter 1: Unit 1C.4). For a review of inhibitors of embryonic stem cell differentiation, see Buchr et al, 2003, Genesis of embryonic stem cells, Philosophical Transactions of the Royal Society B: Biological Sciences 358:1397-1402.

Selected cells incorporating the expression construct can be injected into preimplantation embryos. For microinjection, embryonic stem cells or induced pluripotent stem cells are rendered to single cells using a mixture of trypsin and EDTA, followed by resuspension in embryonic stem cell media. Groups of single cells are selected using a finely drawn-out glass needle (20-25 micrometer inside diameter) and introduced through the embryo's zona pellucida and into the blastocysts cavity (blastocoel) using an inverted microscope fitted with micromanipulators. Stem cells can also be injected into early stage embryos (e.g., 2-cell, 4-cell, 8-cell, premorula, or morula). Injection can be assisted with a laser or piezo-pulsed drilled opening in the zona pellucida. Approximately 9-10 selected stem cells (embryonic stem cells or induced pluripotent stem cells) are injected per blastocyst, or 8-cell stage embryo, 6-9 stem cells per 4-cell stage embryo, and about 6 stem cells per 2-cell stage embryo. Following stem cell introduction, embryos are allowed to recover for a few hours at 37° C. in 5% CO₂, 5% O₂ in nitrogen or cultured overnight before transfer into pseudopregnant recipient females. In a further alternative to stem cell injection, stem cells can be aggregated with morula stage embryos. All of these methods are well established and can be used to produce stem cell chimeras. For a more detailed description, see, e.g., Manipulating the Mouse Embryo: A Laboratory Manual, 3^{rd} edition (Nagy, Gertsenstein, Vintersten, and Behringer, Cold Spring Harbor Laboratory Press, Dec. 15, 2002, ISBN-10:0879695919; see also Nagy et al, 1990, Development 110:815-821; Kraus et al, 2010, Genesis 48:394-399; and U.S. Pat. Nos. 7,576,259, 7,659,442, and 7,294,754).

Pseudopregnant embryo recipients are prepared using methods known in the art. Briefly, fertile female animals between 6-8 weeks of age are mated with vasectomized or sterile males to induce a hormonal state conductive to supporting surgically introduced embryos. At 2.5 days post coitum (dpc) up to 15 of the stem cell containing blastocysts are introduced into the uterine horn very near to the uterus-oviduct junction. For early stage embryos and morula, such embryos are either cultured in vitro into blastocysts or implanted into the oviducts of 0.5 dpc or 1.5 dpc pseudopregnant females according to the embryo stage. Chimeric pups from the implanted embryos are born 16-20 days after the transfer depending on the embryo age at implantation. Chimeric males are selected for breeding. Offspring can be analyzed for transmission of the embryonic stem cell genome by coat color and genetic analysis, such as PCR, Southern blot, or nucleic acid sequencing. Protein expression (e.g., of hSCF, hGM-SCF, hIL-3, and/or hCSF1) can be analyzed by protein analysis (Western blot, ELISA) or other functional assays. Offspring expressing the desired genetic modification can be intercrossed to create non-human animals homozygous for the genetic modification. The genetically modified mice can be crossed with immunodeficient mice to create a congenic immunodeficient strain genetically modified to express hSCF, hGM-SCF, hIL-3, and hCSF1.

A nucleotide sequence encoding a protein to be expressed can be targeted into a specific locus of the stem cell genome that is known to result in reliable expression such as the Hprt or the ROSA26 locus. For targeted transgenics, a targeting construct can be made using recombinant DNA techniques. The targeting construct can optionally include 5′ and 3′ sequences that are homologous to the endogenous gene target. The targeting construct can optionally further include a selectable marker such as neomycin phosphotransferase, hygromycin, or puromycin, a nucleic acid encoding hSCF, hGM-SCF, hIL-3, and/or hCSF1, and a polyadenylation signal, for example. To ensure correct transcription and translation of a nucleotide sequence encoding a desired xenogeneic protein, for example, the sequence is either in frame with the endogenous gene locus, or a splice acceptor site and internal ribosome entry site (IRES) sequence are included. Such a targeting construct can be transfected into stem cells, and the stem cells can be screened to detect the homologous recombination event using PCR, Southern blot, or nucleic acid sequencing. Cells with the correct homologous recombination event can be further analyzed for transgene expression by protein analysis, such as by ELISA or Western blot analysis. If desired, the selectable marker can be removed by treating the stem cells with Cre recombinase. After Cre recombinase treatment, the cells are analyzed for the presence of the nucleotide sequence encoding the expression construct or relevant portion thereof. Cells with the correct genomic event can be selected and injected into preimplantation embryos as described above. Chimeric males are selected for breeding. Offspring can be analyzed for transmission of the embryonic stem cell genome by coat color and genetic analysis, such as PCR, Southern blot, or nucleotide sequencing, and can be tested for xenogeneic protein expression such as by protein analysis (Western blot, ELISA) or other functional assays. Offspring expressing the desired genetic modification can be intercrossed to create animals homozygous for the genetic modification. Mice genetically modified to express hSCF, hGM-SCF, hIL-3, and hCSF1 can then be crossed with immunodeficient animals to create a congenic immunodeficient strain genetically modified to express hSCF, hGM-SCF, hIL-3, and hCSF1.

Embodiments of the invention provide a genetically modified immunodeficient QUAD mouse that includes a nucleotide sequence encoding hSCF, a nucleotide sequence encoding hGM-CSF, a nucleotide sequence encoding hIL-3, and a nucleotide sequence encoding hCSF1 in substantially all of their cells, as well as a genetically modified immunodeficient QUAD mouse that includes a nucleotide sequence encoding hSCF, a nucleotide sequence encoding hGM-CSF, a nucleotide sequence encoding hIL-3, and a nucleotide sequence encoding hCSF1 in some, but not all of their cells. One or multiple copies (such as concatamers) of the nucleotide sequence encoding hSCF, the nucleotide sequence encoding hGM-CSF, the nucleotide sequence encoding hIL-3, and/or the nucleotide sequence encoding hCSF1 can be integrated into the genomes of the cells of the immunodeficient QUAD mouse.

Any of various methods can be used to introduce a nucleotide sequence encoding hSCF, a nucleotide sequence encoding hGM-CSF, a nucleotide sequence encoding hIL-3, and a nucleotide sequence encoding hCSF1 into an immunodeficient mouse to produce an immunodeficient QUAD mouse. Such techniques are well-known in the art and include, but are not limited to, pronuclear microinjection and transformation of embryonic stem cells. Methods for generating genetically modified mice that can be used include, but are not limited to, those described in Sundberg and Ichiki, (Eds.), Genetically Engineered Mice Handbook, CRC Press, 2006; Hofker and van Deursen, (Eds.), Transgenic Mouse Methods and Protocols, Humana Press, 2002; Joyner, Gene Targeting: A Practical Approach, Oxford University Press, 2000; Manipulating the Mouse Embryo: A Laboratory Manual, 3^{rd} edition, Cold Spring Harbor Laboratory Press, Dec. 15, 2002, ISBN-10:0879695919; Turksen (Ed.), Embryonic stem cells: methods and protocols in Methods Mol Biol. 2002, 185, Humana Press; Current Protocols in Stem Cell Biology, ISBN: 978047015180; Meyer et al, PNAS USA, 107 (34):15022-26.

Homology-based recombination gene modification strategies can be used to genetically modify an immunodeficient mouse by “knock-in” to introduce a nucleic acid encoding an exogenous protein or proteins e.g., a nucleotide sequence encoding hSCF, a nucleotide sequence encoding hGM-CSF, a nucleotide sequence encoding hIL-3, and a nucleotide sequence encoding hCSF1 into the genome of the immunodeficient mouse, such as homing endonucleases, integrases, meganucleases, transposons, nuclease-mediated processes using a zinc finger nuclease (ZFN), a Transcription Activator-Like (TAL), a Clustered Regularly Interspaced Short Palindromic Repeats (CRISPR)-Cas, or a Drosophila Recombination-Associated Protein (DRAP) approach. See, for example, Cerbini et al., PLOS One. 2015; 10(1):e0116032; Shen et al., PLOS ONE 8(10):e77696; and Wang et al., Protein & Cell, February 2016, Volume 7, Issue 2, pp 152-156.

An immunodeficient QUAD mouse according to aspects of the present invention expresses hSCF, hGM-CSF, hIL-3, and hCSF1, each at a serum concentration of at least about 0.1 pg/mL, 0.5 pg/mL, 1 pg/mL, 2 pg/mL, 5 pg/mL, 10 pg/mL, 15 pg/mL, 20 pg/mL, 25 pg/mL, 30 pg/mL, 40 pg/mL, 50 pg/mL, 60 pg/mL, 75 pg/mL, 100 pg/mL, 150 pg/mL, 200 pg/mL, 250 pg/mL, 300 pg/mL, 400 pg/mL, 500 pg/mL, 600 pg/mL, 750 pg/mL, 1 ng/mL, 2 ng/mL, 5 ng/ml, 10 ng/mL, 15 ng/ml, 20 ng/ml, 25 ng/ml, 30 ng/mL, 40 ng/mL, 50 ng/mL, 60 ng/mL, 75 ng/ml, 100 ng/ml, 150 ng/mL, 200 ng/mL, 250 ng/ml, 300 ng/mL, 400 ng/mL, 500 ng/mL, 600 ng/mL, or 750 ng/mL.

An immunodeficient QUAD mouse according to aspects of the present invention expresses hSCF, hGM-CSF, hIL-3, and hCSF1, each at a serum concentration of about 0.1 pg/mL to about 1000 ng/mL, about 0.1 pg/mL to about 100 ng/mL, about 0.5 pg/mL to about 50 ng/ml, about 1 pg/mL to about 10 ng/mL, about 0.1 pg/mL to about 100 pg/mL, about 1 pg/mL to about 1 ng/ml, about 10 pg/mL to about 10 ng/ml, about 100 pg/mL to about 100 ng/mL, about 0.1 pg/mL to about 10 pg/mL, about 1 pg/mL to about 100 pg/mL, about 10 pg/mL to about 1 ng/ml, about 100 pg/mL to about 10 ng/ml, about 1 ng/mL to about 100 ng/ml, about 0.1 pg/mL to about 1 pg/mL, about 0.5 pg/mL to about 5 pg/mL, about 1 pg/mL to about 10 pg/mL, about 5 pg/mL to about 50 pg/mL, about 10 pg/mL to about 100 pg/mL, about 50 pg/mL to about 500 pg/mL, about 100 pg/mL to about 1 ng/mL, about 500 pg/mL to about 5 ng/ml, about 1 ng/mL to about 10 ng/mL, about 5 ng/ml to about 50 ng/ml, about 10 ng/ml to about 100 ng/mL, about 50 ng/ml to about 500 ng/ml, about 0.5 ng/ml to about 50 ng/ml, about 1 ng/ml to about 25 ng/ml, about 0.5 ng/ml to about 20 ng/ml, about 0.1 ng/mL to about 5 ng/ml, about 0.5 ng/ml to about 10 ng/mL, about 2 ng/ml to about 20 ng/ml, about 1 ng/mL to about 5 ng/ml, or about 2 ng/ml to about 4 ng/mL.

Aspects of the present invention, an immunodeficient QUAD mouse expresses hSCF, hGM-CSF, hIL-3, and hCSF1, each at a serum concentration of about 1 ng/ml to about 10 ng/mL.

### Immunodeficiency

The term “immunodeficient mouse” refers to a mouse characterized by one or more of: a lack of functional immune cells, such as T cells and B cells; a DNA repair defect; a defect in the rearrangement of genes encoding antigen-specific receptors on lymphocytes; and a lack of immune functional molecules such as IgM, IgG1, IgG2a, IgG2b, IgG3 and IgA. Immunodeficient mice can be characterized by one or more deficiencies in a gene involved in immune function, such as Rag1 and Rag2 (Oettinger, M. A et al., Science, 248:1517-1523, 1990; and Schatz, D. G. et al., Cell, 59:1035-1048, 1989) Immunodeficient mice may have any of these or other defects which result in abnormal immune function in the mice.

According to particular aspects, an immunodeficient QUAD mouse has a

defect in its endogenous gene encoding interleukin-2 receptor γ subunit (IL-2RG) which causes the mouse to express a defective endogenous interleukin-2 receptor γ subunit and/or a reduced amount of endogenous interleukin-2 receptor γ subunit, or the mouse may not express an endogenous interleukin-2 receptor γ subunit at all. The immunodeficient QUAD can optionally be IL-2RG null such that it lacks a functional endogenous IL-2RG gene.

In further aspects, a immunodeficient QUAD mouse has a defect in its endogenous gene encoding DNA-dependent protein kinase, catalytic subunit (Prkdc) which causes the mouse to express a defective endogenous DNA-dependent protein kinase, catalytic subunit and/or a reduced amount of endogenous DNA-dependent protein kinase, catalytic subunit, or the mouse may not express endogenous DNA-dependent protein kinase, catalytic subunit at all. The immunodeficient QUAD mouse can optionally be Prkdc null such that it lacks a functional endogenous Prkdc gene).

“Endogenous,” as used herein in relation to genes and the proteins they encode, refers to genes present in the genome of the mouse at their native gene locus.

In various aspects of the present invention, an immunodeficient QUAD mouse is IL-2rg null and/or Prkdc null, lacking functional endogenous IL-2rg and Prkdc genes. The immunodeficient QUAD mouse can optionally include a Prkdc knockout and/or an IL-2rg knockout. In various aspects of the present invention, the immunodeficient QUAD mouse does not express IL-2RG, does not express Prkdc, or does not express both IL-2RG and Prkdc.

In various aspects of the present invention, an immunodeficient QUAD mouse has severe-combined immunodeficiency. The term “severe combined immune deficiency” or “SCID” refers to a condition characterized by the absence or severe reduction of function of T cells and B cells.

Common forms of SCID include: X-linked SCID, which is characterized by gamma chain gene mutations in the IL-2RG gene and the lymphocyte phenotype T(−) B(+) NK(−). and autosomal recessive SCID. Autosomal recessive SCID can be characterized by Jak3 gene mutations and the lymphocyte phenotype T(−) B(+) NK(−), ADA gene mutations and the lymphocyte phenotype T(−) B(−) NK(−), IL-7R alpha-chain mutations and the lymphocyte phenotype T(−) B(+) NK(+), CD3 delta or epsilon mutations and the lymphocyte phenotype T(−) B(+) NK(+), RAG1/RAG2 mutations and the lymphocyte phenotype T(−) B(−) NK(+), Artemis gene mutations and the lymphocyte phenotype T(−) B(−) NK(+), and CD45 gene mutations and the lymphocyte phenotype T(−) B(+) NK(+).

According to aspects of the present invention, an immunodeficient QUAD mouse has the severe combined immunodeficiency mutation (Prkdc^{scid}), commonly referred to as the scid mutation. The scid mutation is well-known and located on mouse chromosome 16 as described in Bosma et al, 1989, Immunogenetics 29:54-56. Mice homozygous for the scid mutation are characterized by an absence of functional T cells and B cells, lymphopenia, hypoglobulinemia, and a normal hematopoetic microenvironment. The scid mutation can be detected, for example, by the detection of markers for the scid mutation using well-known methods such as PCR or flow cytometry.

A genetically modified immunodeficient mouse according to aspects of the present invention has an IL-2 receptor gamma chain deficiency. The term “IL-2 receptor gamma chain deficiency” refers to decreased IL-2 receptor gamma chain. Decreased IL-2 receptor gamma chain can be due to gene deletion or mutation. Decreased IL-2 receptor gamma chain can be detected, for example, by detection of IL-2 receptor gamma chain gene deletion or mutation and/or detection of decreased IL-2 receptor gamma chain expression using well-known methods.

An immunodeficient QUAD mouse according to aspects of the present invention has severe combined immunodeficiency or an IL-2 receptor gamma chain deficiency in combination with severe combined immunodeficiency.

An immunodeficient QUAD mouse according to aspects of the present

invention has the scid mutation or an IL-2 receptor gamma chain deficiency in combination with the scid mutation.

In various aspects of the present invention, the immunodeficient QUAD mouse is a genetically modified NSG mouse, a genetically modified NRG mouse or a genetically modified NOG mouse, wherein the genome of the genetically-modified NSG, NRG or NOG mouse includes a nucleotide sequence encoding hSCF, a nucleotide sequence encoding hGM-CSF, a nucleotide sequence encoding hIL-3, and a nucleotide sequence encoding hCSF1, wherein the mouse expresses hSCF, hGM-CSF, hIL-3, and hCSF1 proteins.

The terms “NOD scid gamma,” “NOD-scid IL2rg^{null}” and “NSG” are used interchangeably herein to refer to a well-known immunodeficient mouse strain NOD.Cg-Prkdc^{scid} IL-2rg^{tm1Wjl}/SzJ, described in detail in Shultz LD et al, 2005, J. Immunol, 174:6477-89. NSG mice combine multiple immune deficits from the NOD/ShiLtJ background, the severe combined immune deficiency (scid) mutation, and a complete knockout of the interleukin-2 receptor gamma chain. As a result, NSG mice lack mature mouse T, B, and NK cells, and they are deficient in multiple mouse cytokine signaling pathways. NSG mice are characterized by a lack of mouse IL-2R-γ (gamma c) expression, no detectable mouse serum immunoglobulin, no mouse hemolytic complement, no mature mouse T lymphocytes, and no mature mouse natural killer cells.

An NSG mouse that expresses hSCF, hGM-CSF, hIL-3, and hCSF1 is referred to as a “NSG-QUAD” mouse, which indicates that the genome of the genetically-modified NSG, mouse includes a nucleotide sequence encoding hSCF, a nucleotide sequence encoding hGM-CSF, a nucleotide sequence encoding hIL-3, and a nucleotide sequence encoding hCSF1, on the NOD.Cg-Prkdc^{scid} IL-2rg^{tm1Wjl}/SzJ background, wherein the mouse expresses hSCF, hGM-CSF, hIL-3, and hCSF1 proteins.

NRG mice are well-known as NOD.Cg-Rag1^{tm1Mom} Il2rg^{tm1Wjl}/SzJ, described in detail in Shultz LD et al, 2008 Clin Exp Immunol 154(2):270-84.

Advantageously, NSG-QUAD mice exhibit a significantly hightened level of CD33⁺/CD14⁺ human monocytes and CD56⁺ human NK cells in the blood when engrafted with human hematopoictic stem cells when compared with NSG mice.

NSG-QUAD mice also exhibit a surprising property of an enhanced development of human monocytes and NK cells when engrafted with human hematopoietic stem cells. There is disclosed an enhanced functional human immune response to innate stimulation with lipopolysaccharide (LPS) in the HSC-engrafted NSG-QUAD mice, making this mouse model particularly valuable in the study of human immune responses to various stimuli.

An NRG mouse that expresses hSCF, hGM-CSF, hIL-3, and hCSF1 is

referred to as a “NRG-QUAD” mouse, which indicates that the genome of the genetically-modified NRG, mouse includes a nucleotide sequence encoding hSCF, a nucleotide sequence encoding hGM-CSF, a nucleotide sequence encoding hIL-3, and a nucleotide sequence encoding hCSF1, on the NOD.Cg-Rag1^{tm1Mom} Il2rg^{tm1Wjl}/SzJ background, wherein the mouse expresses hSCF, hGM-CSF, hIL-3, and hCSF1 proteins.

NOG mice are well-known as NOD.Cg-Prkdc^{scid} Il2rg^{tm1Sug}/JicTac, described in detail in Ito, M. et al., Blood 100, 3175-3182 (2002).

A NOG mouse that expresses hSCF, hGM-CSF, hIL-3, and hCSF1 is referred to as a “NOG-QUAD” mouse, which indicates that the genome of the genetically-modified NOG, mouse includes a nucleotide sequence encoding hSCF, a nucleotide sequence encoding hGM-CSF, a nucleotide sequence encoding hIL-3, and a nucleotide sequence encoding hCSF1, on the NOD.Cg-Prkdc^{scid} Il2rg^{tm1Sug}/JicTac background.

### Hematopoietic Stem Cell Xenografts

Various aspects of the invention relate to administering xenogeneic

hematopoietic stem cells (HSC) to an immunodeficient QUAD mouse, producing an immunodeficient QUAD mouse having engrafted HSC.

The xenogeneic HSC are human HSC according to aspects of the present invention.

The term “xenogeneic HSC” as used herein refers to multipotent stem cells expressing c-Kit receptor. Examples of multipotent stem cells expressing c-Kit receptor include, but are not limited to, hematopoietic stem cells, also known as hemocytoblasts. C-Kit receptor is well-known in the art, for example as described in

Vandenbark GR et al., 1992, Cloning and structural analysis of the human c-kit gene, Oncogene 7(7): 1259-66; and Edling C E, Hallberg B, 2007, c-Kit—a hematopoietic cell essential receptor tyrosine kinase, Int. J. Biochem. Cell Biol. 39(11):1995-8.

Isolation of xenogeneic HSC, administration of the xenogeneic HSC to a host mouse and methods for assessing engraftment in the host mouse thereof are well-known in the art.

Hematopoietic stem cells for administration to an immunodeficient QUAD mouse can be obtained from any tissue containing HSC such as, but not limited to, umbilical cord blood, bone marrow, GM-CSF-mobilized peripheral blood and fetal liver.

Xenogeneic HSC can be administered into newborn mice by administration via various routes, such as, but not limited to, into the heart, liver and/or facial vein. Xenogeneic HSC can be administered into adult mice by various routes, such as, but not limited to, administration into the tail vein, into the femur bone marrow cavity or into the spleen. In a further example, fetal liver containing the xenogeneic HSC can be engrafted under the renal capsule.

Administering xenogeneic cells to a mouse can include administering a composition comprising xenogeneic cells to the mouse. The composition can further include, for example, water, a tonicity-adjusting agent (e.g., a salt such as sodium chloride), a pH buffer (e.g., citrate), and/or a sugar (e.g., glucose).

Engraftment of xenogeneic hematopoietic stem cells in immunodeficient animals is characterized by the presence of differentiated xenogeneic hematopoietic cells in the immunodeficient QUAD mice. Engraftment of xenogeneic HSC can be assessed by any of various methods, such as, but not limited to, flow cytometric analysis of cells in the animals to which the xenogeneic HSC are administered at one or more time points following the administration of HSC.

Exemplary methods for isolation of xenogeneic HSC, administration of the xenogeneic HSC to a host mouse and methods for assessing engraftment thereof are described herein and in T. Pearson et al., Curr. Protoc. Immunol. 81:15.21.1-15.21.21, 2008; Ito, M. et al, Blood 100:3175-3182; Traggiai, E. et al, Science 304:104-107; Ishikawa, F. et al, Blood 106:1565-1573; Shultz, L. D. et al, J. Immunol. 174:6477-6489; Holyoake TL et al, Exp Hematol., 1999, 27(9):1418-27.

According to aspects of the present invention, the xenogeneic HSC administered to an immunodeficient QUAD mouse are isolated from an original source material to obtain a population of cells enriched in HSCs. The isolated xenogeneic HSCs may or may not be pure. According to aspects, xenogeneic HSCs are purified by selection for a cell marker, such as CD34. According to aspects, administered xenogencic HSCs are a population of cells in which CD34⁺ cells constitute about 1-100% of total cells, although a population of cells in which CD34⁺ cells constitute fewer than about 1% of total cells can be used. According to embodiments, administered xenogeneic HSCs are T cell depleted cord blood cells in which CD34⁺ cells make up about 1-3% of total cells, lineage depleted cord blood cells in which CD34⁺ cells make up about 50% of total cells, or CD34⁺ positively selected cells in which CD34⁺ cells make up about 90% of total cells.

The number of xenogeneic HSCs administered is not considered limiting with regard to generation of a xenogeneic hematopoietic and immune system in an immunodeficient QUAD mouse. A single xenogeneic HSC can generate a hematopoietic and immune system in a host immunodeficient QUAD mouse. Thus, the number of administered xenogeneic HSCs is generally in the range of about 1×10³ to 1×10⁶ (1,000 to 1,000,000) CD34⁺ cells where the recipient is a mouse, although more or fewer can be used.

Thus, a method according to aspects of the present invention can include administering about 10³ (1000) to about 10⁶ (1,000,000), about 10³ to about 10⁵, about 10⁴ to about 10⁶, about 10⁵ to about 10⁷, about 1×10³ to about 1×10⁴, about 5×10³ to about 5×10⁴, about 1×10⁴ to about 1×10⁵, about 5×10⁴ to about 5×10⁵, about 1×10⁵ to about 1×10⁶, about 5×10⁵ to about 5×10⁶, about 1×10⁶ to about 1×10⁷, about 2×10⁴ to about 5×10⁵, or about 5×10⁴ to about 2×10⁵ xenogeneic HSC, such as human hematopoietic stem cells, to the immunodeficient QUAD mouse. The method can include administering at least about 1×10², about 2×10², about 3×10², about 4×10², about 5×10², about 6×10², about 7×10², about 8×10², about 9×10², about 1×10³, about 2×10³, about 3×10³, about 4×10³, about 5×10³, about 6×10³, about 7×10³, about 8×10³, about 9×10³, about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, or about 1×10⁷ xenogeneic HSC, such as human hematopoietic stem cells, to the immunodeficient QUAD mouse. The method can include administering about 1×10², about 2×10², about 3×10², about 4×10², about 5×10², about 6×10², about 7×10², about 8×10², about 9×10², about 1×10³, about 2×10³, about 3×10³, about 4×10³, about 5×10³, about 6×10³, about 7×10³, about 8×10³, about 9×10³, about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, or about 1×10⁷ xenogeneic HSC, such as human HSC, to the immunodeficient QUAD mouse. Those of ordinary skill will be able to determine a number of xenogeneic cells that should be administered to a specific mouse using no more than routine experimentation.

Engraftment is successful where xenogenic HSCs and/or cells differentiated from the xenogencic HSCs in the recipient immunodeficient QUAD mouse are detected at a time when the majority of any administered non-HSC have degenerated. Detection of differentiated xenogeneic HSC cells can be achieved by detection of xenogeneic DNA in the recipient immunodeficient QUAD mouse or detection of intact xenogeneic HSCs and cells differentiated from the xenogeneic HSCs, for example. Serial transfer of CD34⁺ cells into a secondary recipient and engraftment of a xenogeneic hematopoietic system is a further test of HSC engraftment in the primary recipient. Engraftment can be detected by flow cytometry as 0.05% or greater xenogeneic CD45⁺ cells in the blood, spleen or bone marrow of the recipient immunodeficient QUAD mouse at 6-12 weeks after administration of the xenogeneic HSC.

Engraftment of xenogeneic haematopoietic stem cells (HSC) in an immunodeficient QUAD mouse according to aspects of the present invention includes “conditioning” of the immunodeficient QUAD mouse prior to administration of the xenogeneic HSC, for example by sub-lethal irradiation of the recipient animal with high frequency electromagnetic radiation, or gamma radiation, or treatment with a radiomimetic drug such as busulfan or nitrogen mustard. Conditioning is believed to reduce numbers of host hematopoietic cells, create appropriate microenvironmental factors for engraftment of xenogeneic HSC, and/or create microenvironmental niches for engraftment of xenogeneic HSC. Standard methods for conditioning are known in the art, such as described herein and in J. Hayakawa et al, 2009, Stem Cells, 27(1):175-182.

Methods are provided according to aspects of the present invention which include administration of xenogeneic HSC to an immunodeficient QUAD mouse without “conditioning” the immunodeficient QUAD mouse prior to administration of the xenogeneic HSC. Methods are provided according to aspects of the present invention which include administration of xenogeneic HSC to an immunodeficient QUAD mouse without “conditioning” by radiation or radiomimetic drugs of the immunodeficient QUAD mouse prior to administration of the xenogeneic HSC.

Various aspects of the invention relate to administering xenogeneic hematopoietic stem cells (HSC) to an immunodeficient QUAD mouse, producing an immunodeficient QUAD mouse having engrafted HSC. The engrafted xenogeneic HSC differentiate to produce an immunodeficient QUAD mouse with a xenogeneic immune system or components thereof.

Administration of xenogeneic HSC to an immunodeficient QUAD mouse results in the production of differentiated xenogeneic cells of hematopoietic lineage in the mouse. For example, administration of xenogeneic HSC to an immunodeficient QUAD mouse results in the production of xenogeneic myeloid-lineage and xenogeneic lymphoid-lineage cells by the mouse such as xenogeneic CD33⁺ myeloid cells, xenogeneic myeloid progenitor cells, xenogencic mast cells, xenogeneic myeloid dendritic cells, xenogeneic B cells, xenogeneic T cells, xenogeneic T helper cells, xenogeneic cytotoxic T cells, and/or xenogeneic Treg cells. According to aspects of the present invention, human HSC are administered to an immunodeficient QUAD mouse which differentiate such that the mouse includes one or more of: human CD33⁺ myeloid cells, human myeloid progenitor cells, human mast cells, human myeloid dendritic cells, human B cells, human T cells, human T helper cells, human cytotoxic T cells, and/or human T_{reg} cells.

Administration of xenogencic HSC to an immunodeficient QUAD mouse results in the production of differentiated xenogeneic cells of hematopoietic lineage that can be characterized by flow cytometry. For example, administration of human HSC to an immunodeficient QUAD mouse results in the production of differentiated human cells of hematopoietic lineage that can be characterized by flow cytometry, including human leukocytes selected from 1, 2, 3, 4, 5, 6, 7, 8, 9, 10, or all of CD45⁺, CD20⁺, CD20⁺CD45⁺, CD3⁺, CD3⁺CD45⁺, CD33⁺, CD33⁺CD45⁺, CD14⁺, CD14⁺CD45⁺, CD56⁺, and CD56⁺CD45⁺ human leukocytes.

According to aspects disclosed herein, administration of human HSC to an immunodeficient QUAD mouse results in the production of differentiated human cells of hematopoietic lineage such as human CD45⁺ leukocytes, wherein at least about 20%, 25%, 30%, 35%, 40%, 45%, 50%, 55%, 60%, or more of the human CD45⁺ leukocytes of the mouse are CD3⁺CD45⁺ leukocytes; at least about 10%, 15%, 20%, 25%, 30%, 35%, or more of the human CD45⁺ leukocytes of the mouse are CD33⁺CD45⁺ leukocytes; at least about 5%, 10%, 15%, 20%, or more of the human CD45⁺ leukocytes of the mouse are CD14⁺CD45⁺ leukocytes; and/or at least about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, or more of the human CD45⁺ leukocytes of the mouse are CD56⁺CD45⁺ leukocytes (e.g., in the absence of an immunological challenge such as a tumor or antigen challenge).

According to aspects disclosed herein, administration of human HSC to an immunodeficient QUAD mouse results in the production of differentiated human cells of hematopoietic lineage such as human leukocytes, wherein at least about 20%, 25%, 30%, 35%, 40%, 45%, 50%, 55%, 60%, or more of the human leukocytes of the mouse are either cytotoxic T-cells or T-helper cells; at least about 10%, 15%, 20%, 25%, 30%, 35%, or more of the human leukocytes of the mouse are myeloid-lineage cells; at least about 5%, 10%, 15%, 20%, or more of the human leukocytes of the mouse are macrophages; and/or at least about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, or more of the human leukocytes of the mouse are natural killer cells (e.g., in the absence of an immunological challenge such as a tumor or antigen challenge).

According to aspects disclosed herein, administration of xenogeneic HSC to an immunodeficient QUAD mouse results in the production of xenogeneic cytokines by the mouse such as interleukin-8, interleukin-1β, tumor-necrosis factor, interleukin-12p70, and/or interleukin-6. For example, administration of human HSC to an immunodeficient QUAD mouse results in the production of 1, 2, 3, 4, or all of human interleukin-8, human interleukin-1β, human tumor-necrosis factor, human interleukin-12p70, and human interleukin-6.

According to aspects disclosed herein, administration of xenogeneic HSC to an immunodeficient QUAD mouse results in the production of xenogeneic cytokines in response to an immune challenge, e.g., at a detectable level and/or at a level capable of mediating a xenogeneic immune response against the challenge. According to aspects disclosed herein, administration of human HSC to an immunodeficient QUAD mouse results in at least about 0.1 pg/mL, 0.5 pg/mL, 1 pg/mL, 2 pg/mL, 5 pg/mL, 10 pg/mL, 15 pg/mL, 20 pg/mL, 25 pg/mL, 30 pg/mL, 40 pg/mL, 50 pg/mL, 60 pg/mL, 75 pg/mL, 100 pg/mL, 150 pg/mL, 200 pg/mL, 250 pg/mL, 300 pg/mL, 400 pg/mL, 500 pg/mL, 600 pg/mL, 750 pg/mL, 1 ng/mL, 2 ng/mL, 5 ng/ml, 10 ng/ml, 15 ng/mL, or more serum human interleukin-8 in the mouse. According to aspects disclosed herein, administration of human HSC to an immunodeficient QUAD mouse results in the production of at least about 0.1 pg/mL, 0.5 pg/mL, 1 pg/mL, 2 pg/mL, 5 pg/mL, 10 pg/mL, 15 pg/mL, 20 pg/mL, 25 pg/mL, 30 pg/mL, 40 pg/mL, 50 pg/mL, 60 pg/mL, 75 pg/mL, 100 pg/mL, 150 pg/mL, 200 pg/mL, 250 pg/mL, or more serum human interleukin-1β in the mouse. According to aspects disclosed herein, administration of human HSC to an immunodeficient QUAD mouse results in at least about 0.1 pg/mL, 0.5 pg/mL, 1 pg/mL, 2 pg/mL, 5 pg/mL, 10 pg/mL, 15 pg/mL, 20 pg/mL, 25 pg/mL, 30 pg/mL, 40 pg/mL, 50 pg/mL, 60 pg/mL, 75 pg/mL, 100 pg/mL, 150 pg/mL, 200 pg/mL, 250 pg/mL, 300 pg/mL, 400 pg/mL, 500 pg/mL, 600 pg/mL, 750 pg/mL, 1 ng/mL, 2 ng/mL, 5 ng/mL, 10 ng/mL, 15 ng/ml, 20 ng/ml, 25 ng/ml, 30 ng/ml, 40 ng/ml, 50 ng/ml, or more serum human tumor-necrosis factor in the mouse. According to aspects disclosed herein, administration of human HSC to an immunodeficient QUAD mouse results in at least about 0.1 pg/mL, 0.5 pg/mL, 1 pg/mL, 2 pg/mL, 5 pg/mL, 10 pg/mL, 15 pg/mL, 20 pg/mL, 25 pg/mL, 30 pg/mL, 40 pg/mL, 50 pg/mL, 60 pg/mL, 75 pg/mL, 100 pg/mL, or more serum human interleukin-12p70 in the mouse. According to aspects disclosed herein, administration of human HSC to an immunodeficient QUAD mouse results in at least about 0.1 pg/mL, 0.5 pg/mL, 1 pg/mL, 2 pg/mL, 5 pg/mL, 10 pg/mL, 15 pg/mL, 20 pg/mL, 25 pg/mL, 30 pg/mL, 40 pg/mL, 50 pg/mL, 60 pg/mL, 75 pg/mL, 100 pg/mL, 150 pg/mL, 200 pg/mL, 250 pg/mL, 300 pg/mL, 400 pg/mL, 500 pg/mL, 600 pg/mL, 750 pg/mL, 1 ng/mL, 2 ng/mL, 5 ng/mL, 10 ng/ml, 15 ng/ml, 20 ng/mL, 25 ng/ml, 30 ng/mL, or more serum human interleukin-6 in the mouse.

According to aspects disclosed herein, administration of human HSC to an immunodeficient QUAD mouse results in about 0.1 pg/mL to about 1000 ng/ml, about 0.1 pg/mL to about 100 ng/mL, about 0.5 pg/mL to about 50 ng/ml, about 1 pg/mL to about 10 ng/ml, about 0.1 pg/mL to about 100 pg/mL, about 1 pg/mL to about 1 ng/mL, about 10 pg/mL to about 10 ng/ml, about 100 pg/mL to about 100 ng/ml, about 0.1 pg/mL to about 10 pg/mL, about 1 pg/mL to about 100 pg/mL, about 10 pg/mL to about 1 ng/ml, about 100 pg/mL to about 10 ng/ml, about 1 ng/ml to about 100 ng/ml, about 0.1 pg/mL to about 1 pg/mL, about 0.5 pg/mL to about 5 pg/mL, about 1 pg/mL to about 10 pg/mL, about 5 pg/mL to about 50 pg/mL, about 10 pg/mL to about 100 pg/mL, about 50 pg/mL to about 500 pg/mL, about 100 pg/mL to about 1 ng/ml, about 500 pg/mL to about 5 ng/ml, about 1 ng/ml to about 10 ng/ml, about 5 ng/ml to about 50 ng/ml, about 10 ng/ml to about 100 ng/ml, about 50 ng/ml to about 500 ng/ml, about 0.5 ng/ml to about 50 ng/ml, about 1 ng/ml to about 25 ng/ml, about 0.5 ng/ml to about 20 ng/mL, about 0.1 ng/mL to about 5 ng/ml, about 0.5 ng/ml to about 10 ng/mL, or about 2 ng/ml to about 20 ng/mL serum human interleukin-8 in the mouse.

According to aspects disclosed herein, administration of human HSC to an immunodeficient QUAD mouse results in about 0.1 pg/mL to about 1000 ng/ml, about 0.1 pg/mL to about 100 ng/ml, about 0.5 pg/mL to about 50 ng/ml, about 1 pg/mL to about 10 ng/ml, about 0.1 pg/mL to about 100 pg/mL, about 1 pg/mL to about 1 ng/ml, about 10 pg/mL to about 10 ng/ml, about 100 pg/mL to about 100 ng/ml, about 0.1 pg/mL to about 10 pg/mL, about 1 pg/mL to about 100 pg/mL, about 10 pg/mL to about 1 ng/ml, about 100 pg/mL to about 10 ng/ml, about 1 ng/ml to about 100 ng/ml, about 0.1 pg/mL to about 1 pg/mL, about 0.5 pg/mL to about 5 pg/mL, about 1 pg/mL to about 10 pg/mL, about 5 pg/mL to about 50 pg/mL, about 10 pg/mL to about 100 pg/mL, about 50 pg/mL to about 500 pg/mL, or about 100 pg/mL to about 1 ng/mL serum human interleukin-1β in the mouse.

According to aspects disclosed herein, administration of human HSC to an immunodeficient QUAD mouse results in about 0.1 pg/mL to about 1000 ng/ml, about 0.1 pg/mL to about 100 ng/mL, about 0.5 pg/mL to about 50 ng/ml, about 1 pg/mL to about 10 ng/ml, about 0.1 pg/mL to about 100 pg/mL, about 1 pg/mL to about 1 ng/ml, about 10 pg/mL to about 10 ng/ml, about 100 pg/mL to about 100 ng/ml, about 0.1 pg/mL to about 10 pg/mL, about 1 pg/mL to about 100 pg/mL, about 10 pg/mL to about 1 ng/mL, about 100 pg/mL to about 10 ng/ml, about 1 ng/ml to about 100 ng/ml, about 0.1 pg/mL to about 1 pg/mL, about 0.5 pg/mL to about 5 pg/mL, about 1 pg/mL to about 10 pg/mL, about 5 pg/mL to about 50 pg/mL, about 10 pg/mL to about 100 pg/mL, about 50 pg/mL to about 500 pg/mL, about 100 pg/mL to about 1 ng/mL, about 500 pg/mL to about 5 ng/ml, about 1 ng/ml to about 10 ng/ml, about 5 ng/ml to about 50 ng/ml, about 10 ng/ml to about 100 ng/ml, about 50 ng/ml to about 500 ng/ml, about 0.5 ng/ml to about 50 ng/ml, about 1 ng/ml to about 25 ng/ml, or about 0.5 ng/mL to about 20 ng/mL serum human tumor-necrosis factor in the mouse.

According to aspects disclosed herein, administration of human HSC to an immunodeficient QUAD mouse results in about 0.1 pg/mL to about 1000 ng/ml, about 0.1 pg/mL to about 100 ng/ml, about 0.5 pg/mL to about 50 ng/ml, about 1 pg/mL to about 10 ng/ml, about 0.1 pg/mL to about 100 pg/mL, about 1 pg/mL to about 1 ng/ml, about 10 pg/mL to about 10 ng/ml, about 100 pg/mL to about 100 ng/ml, about 0.1 pg/mL to about 10 pg/mL, about 1 pg/mL to about 100 pg/mL, about 10 pg/mL to about 1 ng/ml, about 100 pg/mL to about 10 ng/ml, about 0.1 pg/mL to about 1 pg/mL, about 0.5 pg/mL to about 5 pg/mL, about 1 pg/mL to about 10 pg/mL, about 5 pg/mL to about 50 pg/mL, about 10 pg/mL to about 100 pg/mL, or about 50 pg/mL to about 500 pg/mL serum human interleukin-12p70 in the mouse.

According to aspects disclosed herein, administration of human HSC to an immunodeficient QUAD mouse results in about 0.1 pg/mL to about 1000 ng/ml, about 0.1 pg/mL to about 100 ng/ml, about 0.5 pg/mL to about 50 ng/ml, about 1 pg/mL to about 10 ng/mL, about 0.1 pg/mL to about 100 pg/mL, about 1 pg/mL to about 1 ng/mL, about 10 pg/mL to about 10 ng/ml, about 100 pg/mL to about 100 ng/ml, about 0.1 pg/mL to about 10 pg/mL, about 1 pg/mL to about 100 pg/mL, about 10 pg/mL to about 1 ng/ml, about 100 pg/mL to about 10 ng/ml, about 1 ng/ml to about 100 ng/ml, about 0.1 pg/mL to about 1 pg/mL, about 0.5 pg/mL to about 5 pg/mL, about 1 pg/mL to about 10 pg/mL, about 5 pg/mL to about 50 pg/mL, about 10 pg/mL to about 100 pg/mL, about 50 pg/mL to about 500 pg/mL, about 100 pg/mL to about 1 ng/ml, about 500 pg/mL to about 5 ng/ml, about 1 ng/mL to about 10 ng/mL, about 5 ng/ml to about 50 ng/ml, about 10 ng/ml to about 100 ng/ml, about 50 ng/ml to about 500 ng/ml, about 0.5 ng/ml to about 50 ng/ml, about 1 ng/ml to about 25 ng/mL, about 0.5 ng/ml to about 20 ng/mL, about 0.1 ng/ml to about 5 ng/ml, about 0.5 ng/ml to about 10 ng/mL, or about 2 ng/ml to about 20 ng/ml serum human interleukin-6 in the mouse.

According to aspects disclosed herein, administration of human HSC to an immunodeficient QUAD mouse results in about 5-15 ng/ml serum human interleukin-8, about 5-250 pg/mL scrum human interleukin-1β, about 10-50 ng/ml serum human tumor-necrosis factor, about 20-100 pg/mL serum human interleukin-12p70, and/or about 15-30 ng/mL serum human interleukin-6 (e.g., when challenged with about 50-250 ng intravenous lipopolysaccharide) in the mouse.

According to aspects disclosed herein, administration of xenogeneic HSC to an immunodeficient QUAD mouse results in production of xenogeneic leukocytes that migrate into a tumor, such as xenogeneic tumor-infiltrating lymphocytes and xenogeneic myeloid-derived suppressor cells, in the immunodeficient QUAD mouse. According to aspects disclosed herein, administration of human HSC to an immunodeficient QUAD mouse results in production of human leukocytes that migrate into a tumor, such as human tumor-infiltrating lymphocytes and human myeloid-derived suppressor cells, in the immunodeficient QUAD mouse. For example, such tumor-infiltrating leukocytes are selected from CD20⁺, CD3⁺, CD33⁺, CD14⁺, and CD56⁺ leukocytes.

### Tumor Xenograft

Various aspects of the invention relate to administering xenogeneic tumor cells to an immunodeficient QUAD mouse.

Xenogeneic tumor cells administered to immunodeficient QUAD mice of the present invention can be any of various tumor cells, including but not limited to, cells of a tumor cell line and primary tumor cells. The xenogeneic tumor cells may be derived from any of various organisms, preferably mammalian, including human, non-human primate, rat, guinea pig, rabbit, cat, dog, horse, cow, goat, pig and sheep.

According to specific aspects of the present invention, the xenogeneic tumor cells are human tumor cells. According to specific aspects of the present invention, the human tumor cells are present in a sample obtained from the human, such as, but not limited to, in a blood sample, tissue sample, or sample obtained by biopsy of a human tumor.

Tumor cells obtained from a human can be administered directly to an immunodeficient QUAD mouse of the present invention or may be cultured in vitro prior to administration to the immunodeficient QUAD mouse.

As used herein, the term “tumor” refers to cells characterized by unregulated growth including, but not limited to, pre-neoplastic hyperproliferation, cancer in-situ, neoplasms, metastases and solid and non-solid tumors. Examples of tumors are those caused by cancer include, but are not limited to, lymphoma, leukemia, squamous cell cancer, small-cell lung cancer, non-small cell lung cancer, adenocarcinoma of the lung, squamous carcinoma of the lung, cancer of the peritoneum, adrenal cancer, anal cancer, bile duct cancer, bladder cancer, brain cancer, breast cancer, triple negative breast cancer, central or peripheral nervous system cancers, cervical cancer, colon cancer, colorectal cancer, endometrial cancer, esophageal cancer, gall bladder cancer, gastrointestinal cancer, glioblastoma, head and neck cancer, kidney cancer, liver cancer, nasopharyngeal cancer, nasal cavity cancer, oropharyngeal cancer, oral cavity cancer, osteosarcoma, ovarian cancer, pancreatic cancer, parathyroid cancer, pituitary cancer, prostate cancer, retinoblastoma, sarcoma, salivary gland cancer, skin cancer, small intestine cancer, stomach cancer, testicular cancer, thymus cancer, thyroid cancer, uterine cancer, vaginal cancer and vulval cancer.

The tumor cells can include BR1126, MDA-MB-231, TM1149, or BL0293 cells.

Administering the tumor cells to the immunodeficient QUAD mouse can be any method that is suitable as recognized in the art. For example, administration can include administering cells into an organ, body cavity, or blood vessel such as by injection or implantation, such as subcutaneous and/or intraperitoneal implantation. The tumor cells may be administered as a tumor mass, clumps of tumor cells or as dissociated cells.

Tumor cells can be administered by various routes, such as, but not limited to, by subcutaneous injection, intraperitoneal injection or injection into the tail vein.

Engraftment of xenogeneic tumor cells can be assessed by any of various methods, such as, but not limited to, visual inspection of the mouse for signs of tumor formation.

Any of various methods can be used to measure growth of xenogeneic tumors, including but not limited to, measurement in living mice, measurement of tumors excised from living mice or measurement of tumors in situ or excised from dead mice. Measurements can be obtained using a measuring instrument such as a caliper, measurement using one or more imaging techniques such as ultrasonography, computed tomography, positron emission tomography, fluorescence imaging, bioluminescence imaging, magnetic resonance imaging and combinations of any two or more of these or other tumor measurement methods. The number of tumor cells in a sample obtained from a mouse bearing xenogeneic tumor cells can be used to measure tumor growth, particularly for non-solid tumors. For example, the number of non-solid tumor cells in a blood sample can be assessed to obtain a measurement of growth of a non-solid tumor in a mouse.

The number of tumor cells administered is not considered limiting. A single tumor cell can expand into a detectable tumor in the genetically modified immunodeficient animals described herein. The number of administered tumor cells is generally in the range of 1,000-1×10⁶ tumor cells, although more or fewer can be administered.

Thus, a method according to aspects of the present invention can include administering about 10² (100) to about 10⁷ (10,000,000), about 10³ to about 10⁵, about 10⁴ to about 10⁶, about 10⁵ to about 10⁷, about 1×10³ to about 1×10⁴, about 5×10³ to about 5×10⁴, about 1×10⁴ to about 1×10⁵, about 5×10⁴ to about 5×10⁵, about 1×10⁵ to about 1×10⁶, about 5×10⁵ to about 5×10⁶, about 1×10⁶ to about 1×10⁷, about 2×10⁴ to about 5×10⁵, or about 5×10⁴ to about 2×10⁵ xenogeneic tumor cells, such as human tumor cells, to the immunodeficient QUAD mouse. The method can include administering at least about 1×10², about 2×10², about 3×10², about 4×10², about 5×10², about 6×10², about 7×10², about 8×10², about 9×10², about 1×10³, about 2×10³, about 3×10³, about 4×10³, about 5×10³, about 6×10³, about 7×10³, about 8×10³, about 9×10³, about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, or about 1×10⁷ xenogeneic tumor cells, such as human tumor cells, to the immunodeficient QUAD mouse. The method can include administering about 1×10², about 2×10², about 3×10², about 4×10², about 5×10², about 6×10², about 7×10², about 8×10², about 9×10², about 1×10³, about 2×10³, about 3×10³, about 4×10³, about 5×10³, about 6×10³, about 7×10³, about 8×10³, about 9×10³, about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, or about 1×10⁷ xenogeneic tumor cells, such as human tumor cells, to the immunodeficient QUAD mouse. Those of ordinary skill will be able to determine a number of xenogeneic tumor cells that should be administered to a specific mouse using no more than routine experimentation.

According to aspects of the present invention, xenogeneic tumor cells and xenogeneic HSC are administered to an immunodeficient QUAD mouse. The xenogeneic tumor cells and xenogeneic HSC can be administered at the same time or at different times.

According to aspects of the present invention, the tumor cells are derived from the same species as the administered HSC. According to aspects, both the tumor cells and the HSC administered to an immunodeficient QUAD mouse are human cells.

According to aspects of the present invention the administered HSC and tumor cells are human leukocyte antigen (HLA) matched (e.g., MHC Class I matched and/or MHC class II matched). HLA-matching may reduce the likelihood of a graft-versus-graft immune response against HLA cell-surface proteins. An immune response against HLA may falsely suggest that xenogeneic immune cells are successfully targeting xenogeneic cancer cells.

The administered HSC and tumor cells can include at least 2, 3, 4, 5, 6, 7,8, 9, 10, 11, 12, 13, 14, 15, 16, 17, 18, 19, or 20 matching HLA alleles. The administered HSC and tumor cells can include at least 2, 3, 4, 5, 6, 7, or 8 matching HLA alleles selected from the alleles for HLA-A, HLA-B, HLA-C, and HLA-DRB1. Perfect HLA matching is rarely possible without genetic engineering, and perfect HLA matching may be unnecessary. Control experiments, for example, can account for any HLA-mediated graft-versus-graft immune response.

### Assay Methods

Methods and immunodeficient QUAD mice provided according to aspects of the present invention have various utilities such as, in vivo testing of substances directed against human cancer.

Methods for identifying anti-tumor activity of a test substance according to aspects of the present invention include providing an immunodeficient QUAD mouse; administering xenogeneic tumor cells to the immunodeficient QUAD mouse, wherein the xenogeneic tumor cells form a solid or non-solid tumor in the immunodeficient QUAD mouse; administering a test substance to the immunodeficient QUAD mouse; assaying a response of the xenogeneic tumor and/or tumor cells to the test substance, wherein an inhibitory effect of the test substance on the tumor and/or tumor cells identifies the test substance as having anti-tumor activity.

Methods for identifying anti-tumor activity of a test substance according to aspects of the present invention include providing an immunodeficient QUAD mouse, wherein the immunodeficient QUAD mouse has engrafted xenogeneic HSC; administering xenogeneic tumor cells to the immunodeficient QUAD mouse, wherein the xenogeneic tumor cells form a solid or non-solid tumor in the immunodeficient QUAD mouse; administering a test substance to the immunodeficient QUAD mouse; assaying a response of the xenogeneic tumor and/or tumor cells to the test substance, wherein an inhibitory effect of the test substance on the tumor and/or tumor cells identifies the test substance as having anti-tumor activity.

Methods for identifying anti-tumor activity of a test substance according to aspects of the present invention include providing an immunodeficient QUAD mouse, wherein the immunodeficient QUAD mouse has engrafted human HSC; administering human tumor cells to the immunodeficient QUAD mouse, wherein the human tumor cells form a solid or non-solid tumor in the immunodeficient QUAD mouse;

administering a test substance to the immunodeficient QUAD mouse; assaying a response of the human tumor and/or tumor cells to the test substance, wherein an inhibitory effect of the test substance on the tumor and/or tumor cells identifies the test substance as having anti-tumor activity.

An immunodeficient QUAD mouse used in an assay for identifying anti-tumor activity of a test substance according to aspects of the present invention is an NSG-QUAD mouse, an NRG-QUAD mouse or a NOG-QUAD mouse.

The term “inhibitory effect” as used herein refers to an effect of the test substance to inhibit one or more of: tumor growth, tumor cell metabolism and tumor cell division.

Assaying a response of the xenogeneic tumor and/or tumor cells to the test substance includes comparing the response to a standard to determine the effect of the test substance on the xenogeneic tumor cells according to aspects of methods of the present invention, wherein an inhibitory effect of the test substance on the xenogeneic tumor cells identifies the test substance as an anti-tumor composition. Standards are well-known in the art and the standard used can be any appropriate standard. In one example, a standard is a compound known to have an anti-tumor effect. In a further example, non-treatment of a comparable xenogeneic tumor provides a base level indication of the tumor growth without treatment for comparison of the effect of a test substance. A standard may be a reference level of expected tumor growth previously determined in an individual comparable mouse or in a population of comparable mice and stored in a print or electronic medium for recall and comparison to an assay result.

Assay results can be analyzed using statistical analysis by any of various methods to determine whether the test substance has an inhibitory effect on a tumor, exemplified by parametric or non-parametric tests, analysis of variance, analysis of covariance, logistic regression for multivariate analysis, Fisher's exact test, the chi-square test, Student's T-test, the Mann-Whitney test, Wilcoxon signed ranks test, McNemar test, Friedman test and Page's L trend test. These and other statistical tests are well-known in the art as detailed in Hicks, CM, Research Methods for Clinical Therapists: Applied Project Design and Analysis, Churchill Livingstone (publisher); 5th Ed., 2009; and Freund, R J et al., Statistical Methods, Academic Press; 3rd Ed., 2010.

A test substance used in a method of the present invention can be any chemical entity, illustratively including a synthetic or naturally occurring compound or a combination of a synthetic or naturally occurring compound, a small organic or inorganic molecule, a protein, a peptide, a nucleic acid, a carbohydrate, an oligosaccharide, a lipid or a combination of any of these. According to aspects of the present invention, the test substance is an immunotherapeutic.

According to aspects of the present invention, a test substance is an anti-cancer agent. According to aspects of the present invention, the test substance is an anti-cancer immunotherapeutic, such as an anti-cancer antibody or antigen binding fragment thereof.

Anti-cancer agents are described, for example, in Brunton et al. (eds.), Goodman and Gilman's The Pharmacological Basis of Therapeutics, 12th Ed., Macmillan Publishing Co., 2011.

Anti-cancer agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, cobimetinib, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflomithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, vemurafenib, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin.

According to aspects of the present invention, an anti-cancer agent is an anti-cancer antibody. An anti-cancer antibody used can be any antibody effective to inhibit at least one type of tumor, particularly a human tumor. Anti-cancer antibodies include, but are not limited to, 3F8, 8H9, abagovomab, abituzumab, adalimumab, adecatumumab, aducanumab, afutuzumab, alacizumab pegol, alemtuzumab, amatuximab, anatumomab mafenatox, anetumab ravtansine, apolizumab, arcitumomab, ascrinvacumab, atezolizumab, bavituximab, belimumab, bevacizumab, bivatuzumab mertansine, brentuximab vedotin, brontictuzumab, cantuzumab mertansine, cantuzumab ravtansine, capromab pendetide, catumaxomab, cetuximab, citatuzumab bogatox, cixutumumab, clivatuzumab tetraxetan, coltuximab ravtansine, conatumumab, dacetuzumab, dalotuzumab, demcizumab, denintuzumab mafodotin, depatuxizumab mafodotin, durvalumab, dusigitumab, edrecolomab, clotuzumab, emactuzumab, emibetuzumab, enoblituzumab, enfortumab vedotin, enavatuzumab, epratuzumab, ertumaxomab, etaracizumab, farletuzumab, ficlatuzumab, figitumumab, flanvotumab, futuximab, galiximab, ganitumab, gemtuzumab, girentuximab, glembatumumab vedotin, ibritumomab tiuxetan, igovomab, imab362, imalumab, imgatuzumab, indatuximab ravtansine, indusatumab vedotin, inebilizumab, inotuzumab ozogamicin, intetumumab, ipilimumab, iratumumab, isatuximab, labetuzumab, lexatumumab, lifastuzumab vedotin, lintuzumab, lirilumab, lorvotuzumab mertansine, lucatumumab, lumiliximab, lumretuzumab, mapatumumab, margetuximab, matuzumab, milatuzumab, mirvetuximab soravtansine, mitumomab, mogamulizumab, moxctumomab pasudotox, nacolomab tafenatox, naptumomab estafenatox, narnatumab, necitumumab, nesvacumab, nimotuzumab, nivolumab, ocaratuzumab, ofatumumab, olaratumab, onartuzumab, ontuxizumab, oregovomab, oportuzumab monatox, otlertuzumab, panitumumab, pankomab, parsatuzumab, patritumab, pembrolizumab, pemtumomab, pertuzumab, pidilizumab, pinatuzumab vedotin, polatuzumab vedotin, pritumumab, racotumomab, radretumab, ramucirumab, rilotumumab, rituximab, robatumumab, sacituzumab govitecan, samalizumab, seribantumab, sibrotuzumab, siltuximab, sofituzumab vedotin, tacatuzumab tetraxetan, tarextumab, tenatumomab, teprotumumab, tetulomab, tigatuzumab, tositumomab, tovetumab, trastuzumab, tremelimumab, tucotuzumab celmoleukin, ublituximab, utomilumab, vandortuzumab vedotin, vantictumab, vanucizumab, varlilumab, vesencumab, volociximab, vorsetuzumab mafodotin, votumumab, zalutumumab and zatuximab.

According to aspects of the present invention, a test substance is one that specifically binds one or more of: 1) a cell-surface protein such as a cluster of differentiation (CD) cell-surface molecule; 2) an intracellular protein such as a kinase; and 3) an extracellular protein such as a shed cell-surface receptor or the soluble ligand of a cell-surface receptor.

According to aspects of the present invention, a test substance is one that specifically binds a protein that is expressed by leukocytes (e.g., lymphocytes or myeloid-lineage leukocytes). In a further option, a test substance is one that specifically binds a ligand of a leukocyte. In a still further option, a test substance is one that specifically binds a molecule that is expressed by a cancer cell.

According to aspects of the present invention, a test substance is a chemotherapeutic agent or an immunotherapeutic agent. According to aspects of the present invention, a test substance can specifically binds PD-1, PD-L1, or CTLA-4. According to aspects of the present invention, a test substance can be an immune checkpoint inhibitor such as a PD-1 inhibitor, PD-L1 inhibitor, or CTLA-4 inhibitor. According to aspects of the present invention, a test substance is an immune checkpoint inhibitor selected from atezolizumab, avelumab, durvalumab, ipilimumab, nivolumab, pembrolizumab, and an antigen-binding fragment of any one of the foregoing.

The test substance can be administered by any suitable route of administration, such as, but not limited to, oral, rectal, buccal, nasal, intramuscular, vaginal, ocular, otic, subcutaneous, transdermal, intratumoral, intravenous, and intraperitoneal.

A genetically-modified, immunodeficient mouse is provided along with methods of use, wherein the mouse includes (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, and wherein the genetically-modified, immunodeficient mouse expresses hSCF, hGM-CSF, hIL-3, and hCSF1, wherein the genetically-modified, immunodeficient mouse allows engraftment of human hematopoietic stem cells along with engraftment of human-patient derived tumor xenografts and/or human tumor cell lines to enable in vivo investigation of the interactions between the human immune system and human cancer.

Embodiments of inventive compositions and methods are illustrated in the following examples. These examples are provided for illustrative purposes and are not considered limitations on the scope of inventive compositions and methods.

### EXAMPLES

### Example 1

### Analysis of NSG-Quad Mice having a Human Hematopoietic Stem Cell Xenograft

Mice that constitutively express a transgene encoding human colony stimulating factor 1 (hCSF1) were generated in an NSG background using routine techniques and are referred to herein as “NSG-CSF1” mice. To make the NSG-CSF1 mice, a transgene encoding human CSF1 (hCSF1) is designed. Expression of hCSF1 is driven by a human cytomegalovirus promoter/enhancer sequence, and is followed by a human growth hormone cassette and a polyadenylation (polyA) sequence. The hCSF1 transgene is microinjected into fertilized C57BL/6xC3H/HeN oocytes. The resulting founders, carrying the hCSF1transgene are backcrossed to BALB/c-Prkdc^{scid} mice for several generations and subsequently backcrossed to NOD.CB17-Prkdc^{scid} mice for several generations. These mice are backcrossed to NOD.Cg-Prkdc^{scid} Il2rg^{tm1Wjl} mice, and are then intercrossed until all offspring are homozygous for the hCSF1 transgene and the IL2rg targeted mutation. These transgenic mice are bred to NOD.Cg-Prkdc^{scid} Il2rg^{tm1Wjl}/SzJ mice (NSG mice, Jackson Laboratory Stock No. 005557) to establish the colony. Thereafter, females homozygous for Prkdc^{scid}, homozygous for Il2rg^{tm1Wjl} and homozygous for the hCSF1 transgene were bred with males homozygous for Prkdc^{scid} hemizygous for the X-linked Il2rg^{tm1Wjl} and homozygous for the hCSF1 transgene.

NSG-CSF1 are maintained on the NOD.Cg-Prkdc^{scid} Il2rg^{tm1Wjl}/SzJ mouse (Stock No. 005557) background and constitutively produce detectable serum levels of human CSF1.

NSG-SGM3 mice express human transgenes for stem cell factor (hSCF), granulocyte-macrophage-colony stimulating factor (hGM-CSF), and interleukin-3 (hIL-3). NSG-SGM3 mice contain three transgenes, human interleukin-3 (IL-3), human granulocyte/macrophage-stimulating factor (GM-CSF), and human Steel factor (SF) gene, each driven by a human cytomegalovirus promoter/enhancer sequence. To make the NSG-SGM3 mice, three separate transgenes were designed each carrying one of: the human interleukin-3 (IL-3) gene, the human granulocyte/macrophage-stimulating factor (GM-CSF) gene, or human Steel factor (SF) gene. Expression of each gene is driven by a human cytomegalovirus promoter/enhancer sequence, and is followed by a human growth hormone cassette and a polyadenylation (polyA) sequence. The three transgenes were microinjected into fertilized C57BL/6xC3H/HeN oocytes. The resulting founders, carrying all three transgenes (3GS) were backcrossed to BALB/c-Prkdc^{scid} mice for several generations and subsequently backcrossed to NOD.CB17-Prkdc^{scid} mice for at least 11 generations. These mice were bred to NOD.Cg-Prkdc^{scid} Il2rg^{tm1Wjl} mice, and were then intercrossed until all offspring were homozygous for 3GS and the IL2rg targeted mutation. These transgenic mice were bred to NOD.Cg-Prkdc^{scid} Il2rg^{tm1Wjl}/SzJ mice (NSG mice, Jackson Laboratory Stock No. 005557) for one generation to establish the colony. Thereafter, females homozygous for Prkdc^{scid}, homozygous for Il2rg^{tm1Wjl} and homozygous for the 3GS transgenes were bred with males homozygous for Prkdc^{scid}, hemizygous for the X-linked Il2rg^{tm1Wjl} and homozygous for the 3GS transgenes.

NSG-SGM3 are maintained on the NOD.Cg-Prkdc^{scid} Il2rg^{tm1Wjl}/SzJ

mouse (Stock No. 005557) background and constitutively produce human IL-3, GM-CSF, and SCF. NSG-SGM3 can be obtained commercially from The Jackson Laboratory (Maine; Stock No. 013062).

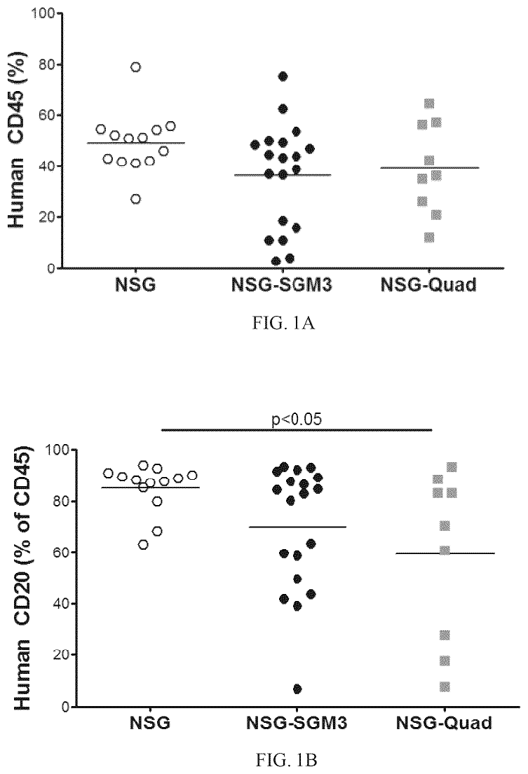

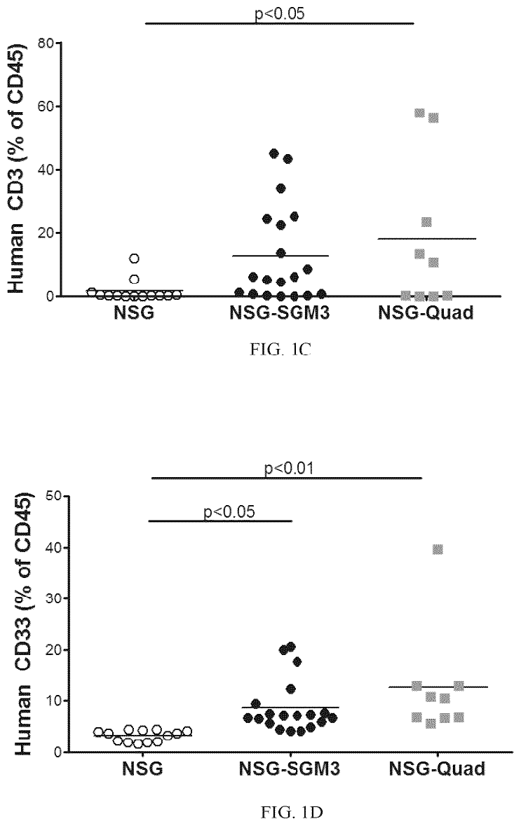

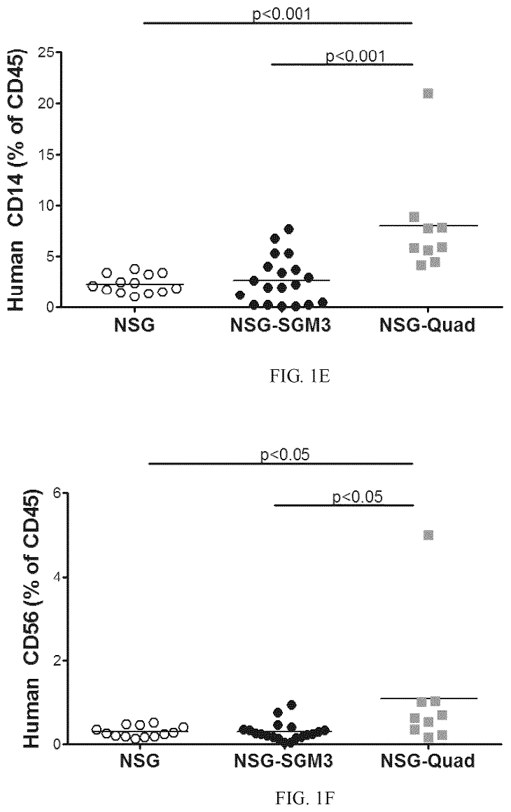

NSG-Quad mice were irradiated with 50 cGy whole body radiation. NSG-SGM3 mice were irradiated with 50 cGy whole body radiation and NSG mice were irradiated with 100 cGy whole body radiation as controls. Each mouse received about 100,000 human CD34⁺ hematopoietic stem cells from the same donor by injection. Blood cells obtained by retro-orbital bleeding at 10 weeks post-engraftment were assessed by flow cytometry for mouse CD45, human CD45, human CD20, human CD3, human CD33, human CD14, and human CD56 (FIG. 1A-F). Live cells were gated using LIVE/DEAD® (ThermFisher Scientific).

NSG-Quad mice displayed fewer numbers of human CD20⁺ cells, increased numbers of human CD3⁺ cells, CD33⁺ cells, CD14⁺ cells, and CD56⁺ cells as a proportion of human CD45⁺ cells relative to NSG mice, which suggests that NSG-Quad mice are better able to differentiate human hematopoietic progenitor cells into T-lymphocytes, macrophages, and natural killer cells than NSG mice (FIG. 1B-F). NSG-Quad mice displayed increased numbers of human CD14⁺ cells and CD56⁺ cells as a proportion of human CD45⁺ cells relative to NSG-SGM3 mice, which suggests that NSG-Quad mice are better able to differentiate human hematopoietic progenitor cells into macrophages (myeloid lineage) and natural killer cells (lymphoid lineage) than NSG-SGM3 mice (FIG. 1E-F). NSG-Quad mice trended toward increased human CD3⁺ cell counts and increased human CD33⁺ cell counts as a proportion of human CD45⁺ cells relative to NSG-SGM3 mice, although these trends did not display statistical significance (FIG. 1C-D). The trend toward increased human CD3⁺ cell counts nevertheless suggests that the addition of CSF1, which is believed to be a myeloid-lineage cytokine, to the NSG-SGM3 mouse had an effect on lymphoid-lineage cells.

### Example 2

### Immune Function of NSG-Quad Mice having a Human Hematopoietic Stem Cell Xenograft

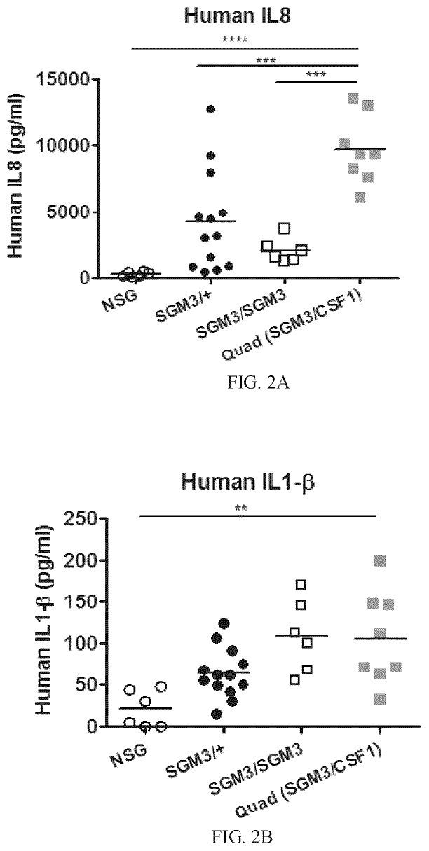

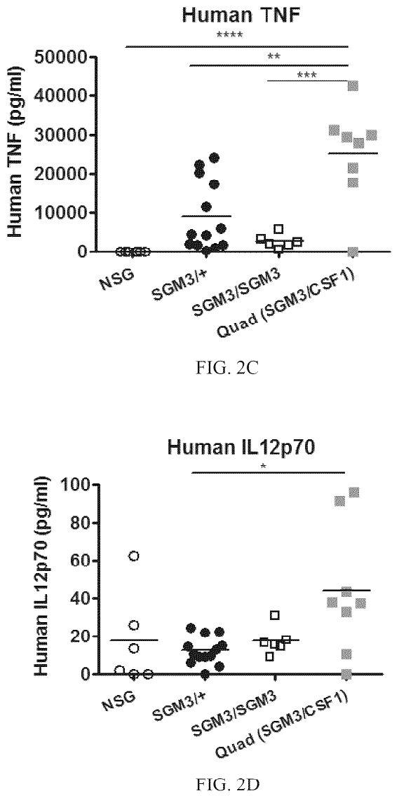

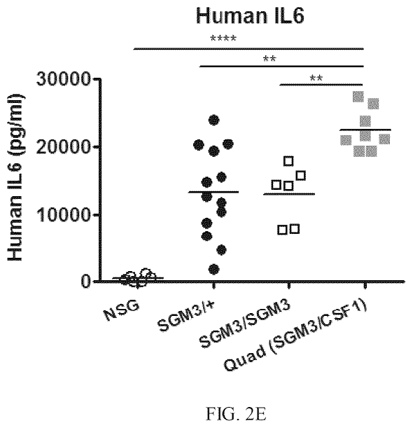

NSG-Quad mice, NSG-SGM3 hemizygous mice, NSG-SGM3 homozygous mice, and NSG mice engrafted with human CD34⁺ cells as described in Example 1 were administered an intravenous injection of 0.15 μg lipopolysaccharide (LPS) 10-weeks post xenograft. Serum was collected 6 hours after the LPS challenge, and human cytokine concentrations were determined using the BD™ Cytometric Bead Array (BD Biosciences). The NSG-Quad mice displayed increased concentrations of macrophage-secreted cytokines relative to NSG-SGM3 hemizygous mice, NSG-SGM3 homozygous mice, and NSG mice (FIG. 2A-E).

### Example 3

### Tumor Growth in NSG-Quad Mice

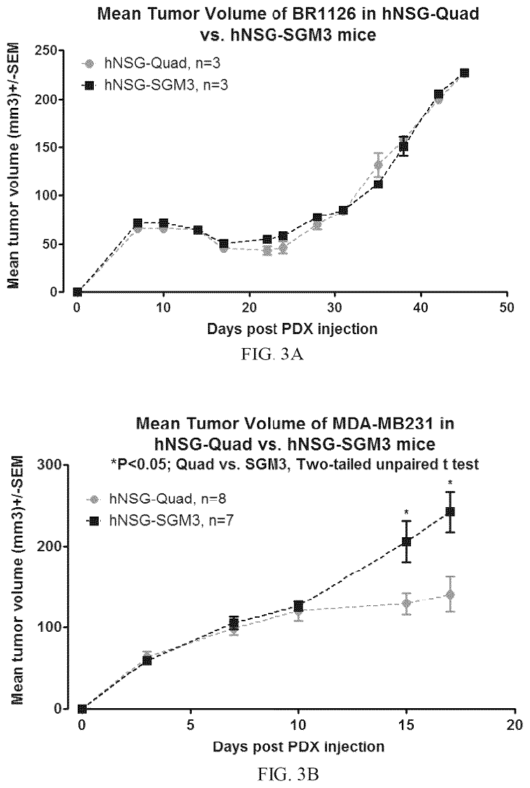

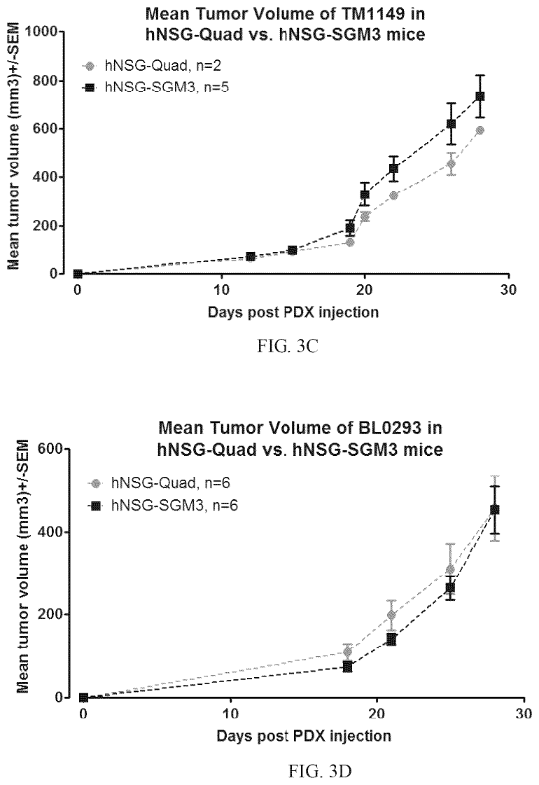

BR1126, MDA-MB-231, TM1149, and BL0293 tumor cells were separately implanted subcutaneously into NSG-Quad and NSG-SGM3 mice at 7-12 weeks post-CD34⁺-cell engraftment (see Example 1). Tumor sizes were measured with calipers. The BR1126, TM1149, and BL0293 tumors displayed similar growth in both the NSG-Quad and NSG-SGM3 mice (FIG. 3A, 3C, 3D). The NSG-Quad mice displayed significantly smaller tumors day 15 after implantation with MDA-MB-231 tumor cells (FIG. 3B).

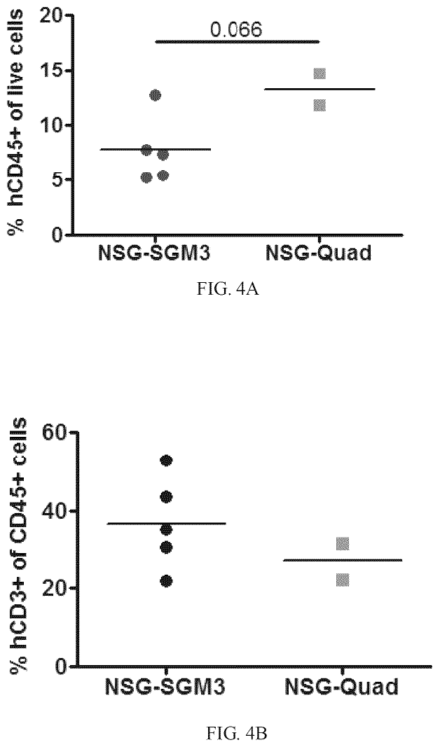

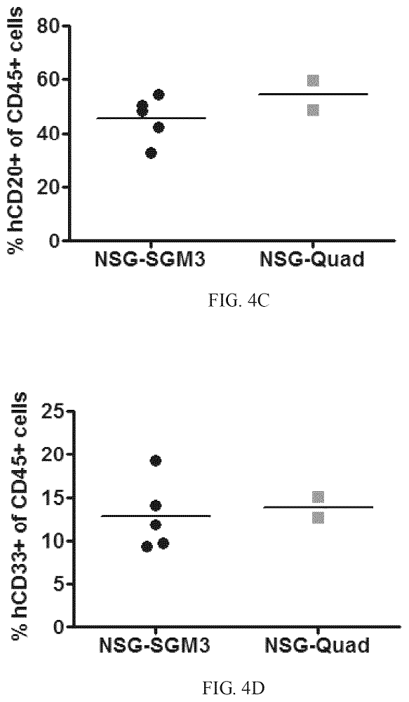

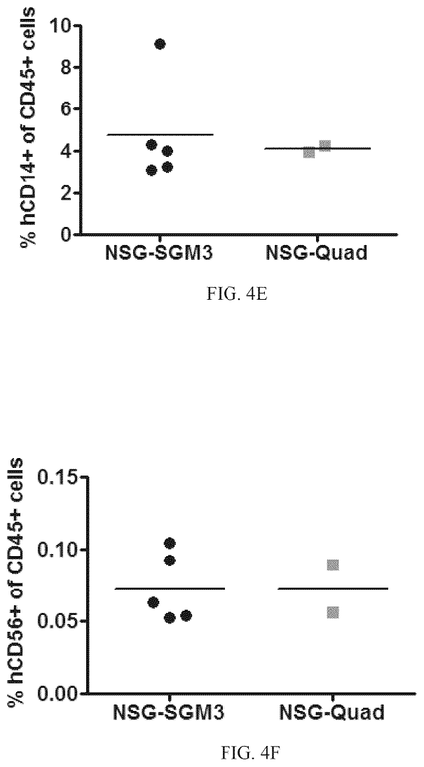

Human TM1149 (breast cancer) tumors were harvested from NSG-Quad and NSG-SGM3 mice at 17-weeks post tumor-cell engraftment, and cells were analyzed by flow cytometry for mouse CD45, human CD45, human CD20, human CD3, human CD33, human CD14, and human CD56 (FIG. 4A-F). Live cells were gated using LIVE/DEAD® (ThermoFisher Scientific). NSG-Quad displayed significantly more intratumoral leukocytes than NSG-SGM3 mice (FIG. 4A). Interestingly, the NSG-Quad mice trended toward increased human CD20⁺ cell counts, although this trend did not display statistical significance (FIG. 4C).

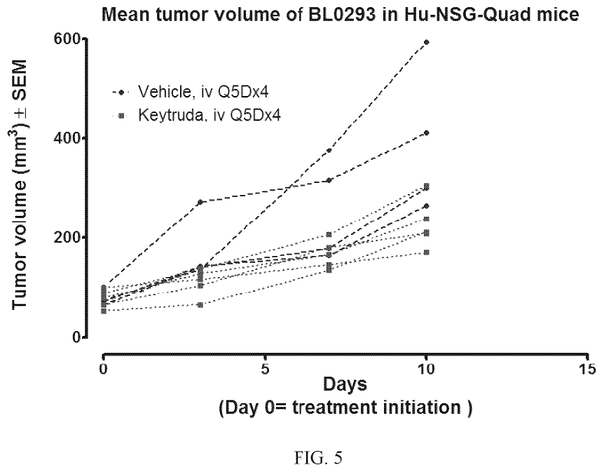

NSG-Quad mice xenografted with human BL0293 tumor cells (bladder cancer cells) were grouped when tumors reached 60-100 mm³, and mice were then administered intravenous pembrolizumab at 10 mg/kg on day 0 followed by 5 mg/kg on days 5, 10, and 15. Tumor sizes were measured by calipers. Pembrolizumab reduced tumor growth rates in NSG-Quad mice (FIG. 5).

### Figures

FIG. 1 consists of 6 panels, labeled FIG. 1A, FIG. 1B, FIG. 1C, FIG. 1D, FIG. 1E, and FIG. 1F. Each of FIGS. 1A, 1B, 1C, 1D, 1E, and 1F is a chart depicting percentages of flow-cytometry-gated blood cells observed in mice. Each data point corresponds to a single mouse. The x-axis labels identify three mouse strains, NSG, NSG-SGM3, and NSG-Quad, each of which is described herein. The y-axis identifies percentages of gated cells. NSG, NSG-SGM3, and NSG-Quad mice were each administered 100,000 human CD34⁺ hematopoietic stem cells, and blood cells were collected for flow cytometry analysis by retro-orbital bleeds 10-weeks post-engraftment.

Statistically significant results are shown as p-values. The NSG-Quad mice displayed statistically significant increases in the relative number of human CD14⁺ cells (macrophages) and human CD56⁺ cells (natural killer cells) relative to NSG-SGM3 mice. Additionally, NSG-Quad mice trended toward increased human CD3⁺ cells (T-lymphocytes) relative to NSG-SGM3 mice, but this observation did not achieve statistical significance.

FIG. 2 consists of 5 panels, labeled FIG. 2A, FIG. 2B, FIG. 2C, FIG. 2D, and FIG. 2E. Each of FIGS. 2A, 2B, 2C, 2D, and 2E is a chart depicting the concentration of cytokines observed in mouse serum. Each data point corresponds to a single mouse. The x-axis labels identify four mouse strains, NSG, NSG-SGM3/+, NSG-SGM3/SGM3, and NSG-Quad, each of which is described herein. The y-axis identifies concentrations of cytokines. NSG, NSG-SGM3/+, NSG-SGM3/SGM3, and NSG-Quad mice were each administered 100,000 human CD34⁺ hematopoietic stem cells followed by 0.15 μg intravenous lipopolysaccharide 10-weeks later. Serum cytokine concentrations were measured 6 hours after lipopolysaccharide administration using the BD™ Cytometric Bead Array for macrophage-associated cytokines. NSG-Quad mice, which constitutively express the human macrophage differentiation cytokine hCSF1, displayed elevated concentrations of macrophage-associated cytokines relative to control animals.

FIG. 3 consists of 6 panels, labeled FIG. 3A, FIG. 3B, FIG. 3C, and FIG. 3D. Each of FIGS. 3A, 3B, 3C, and 3D is a graph depicting the average tumor volume of mice having patient-derived xenografts (PDX). Each data point corresponds to an average tumor volume observed in a group of mice at a specific time-point. Circles ( [symbol] ) correspond to the NSG-Quad group of mice and squares ( [symbol] ) correspond to the NSG-SGM3 group. The x-axis identifies the number of days after a mouse received a patient-derived xenograft. The y-axis identifies the average tumor volume in mm³. Error bars correspond to standard error. Each patient-derived xenograft displayed similar growth in the NSG-Quad group and the NSG-SGM3 group except for the human MDA-MB-231 xenograft, which displayed significantly less growth in the NSG-Quad group relative to the NSG-SGM3 group beginning at day 15 post-engraftment.

FIG. 4 consists of 6 panels, labeled FIG. 4A, FIG. 4B, FIG. 4C, FIG. 4D, FIG. 4E, and FIG. 4F. Each of FIGS. 4A, 4B, 4C, 4D, 4E, and 4F is a chart depicting percentages of flow-cytometry-gated intratumor leukocytes observed in mice. Each data point corresponds to a single mouse. The x-axis labels identify two mouse strains, NSG-SGM3 and NSG-Quad, each of which is described herein. The y-axis identifies percentages of gated cells. NSG-SGM3 and NSG-Quad mice were each administered 100,000 human CD34⁺ hematopoietic stem cells followed by a subcutaneous TM1149 xenograft 7-12 weeks later. Tumors were harvested 17 weeks after the TM1149 xenograft and analyzed by flow cytometry. Statistically significant results are shown as p-values. NSG-Quad mice displayed a statistically significant increase in total human CD45⁺ cells (leukocytes) relative to NSG-SGM3 mice, which suggests that NSG-Quad mice may be superior to NSG-SGM3 mice for studying tumor-infiltrating lymphocyte-dependent cancer immunotherapies. NSG-Quad mice trended toward increased numbers of human CD3⁺ cells (T-lymphocytes) relative to NSG-SGM3 mice, but this observation did not display statistical significance.

FIG. 5 is a graph depicting tumor volumes for NSG-Quad mice that have human CD34⁺ hematopoietic stem cell xenografts and BL0293 patient-derived xenografts and receiving either pembrolizumab (Keytruda®) or vehicle. Each data point corresponds to a single mouse at a specific time-point. Circles ( [symbol] ) correspond to vehicle-treated mice and squares ( [symbol] ) correspond to pembrolizumab-treated mice. The x-axis identifies the number of days after a mouse began receiving pembrolizumab or vehicle. The y-axis identifies the average tumor volume in mm³. NSG-Quad mice treated with pembrolizumab displayed slower tumor growth on average than mice receiving vehicle.

All patents and publications mentioned in this specification are incorporated herein by reference to the same extent as if each individual publication was specifically and individually indicated to be incorporated by reference.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

The mice and methods described herein are presently representative of preferred embodiments, exemplary, and not intended as limitations on the scope of the invention. Modifications thereto and additional uses will occur to those skilled in the art. Such modifications and uses can be made without departing from the scope of the invention as set forth in the claims.

### Items

Item 1. A genetically-modified, immunodeficient mouse, comprising: (a) a nucleotide sequence encoding human stem cell factor (hSCF); (b) a nucleotide sequence encoding human granulocyte-macrophage colony-stimulating factor (hGM-CSF); (c) a nucleotide sequence encoding human interleukin-3 (hIL-3); and (d) a nucleotide sequence encoding human colony-stimulating factor 1 (hCSF1), wherein each of the nucleotide sequences is operably linked to a promoter, and wherein the genetically-modified, immunodeficient mouse expresses hSCF, hGM-CSF, hIL-3, and hCSF1.

Item 2. The genetically-modified, immunodeficient mouse of item 1, wherein the mouse is a genetically modified NOD.Cg-Prkdc^{scid} Il2rg^{tm1Wjl}/SzJ (NSG) mouse; a genetically modified NOD.Cg-Rag1^{tm1Mom} Il2rg^{tm1Wjl}/SzJ (NRG) mouse or a genetically modified NOD.Cg-Prkdc^{scid} Il2rg^{tm1Sus}/JicTac (NOG) mouse.

Item 3. The genetically-modified, immunodeficient mouse of any one of the preceding items, wherein any one or more of the nucleotide sequences encoding hSCF, hGM-CSF, hIL-3, or hCSF1 is operably-linked to a constitutive promoter.

Item 4. The genetically-modified, immunodeficient mouse of any one of the preceding items, further comprising a human hematopoietic stem cell.

Item 5. The genetically-modified, immunodeficient mouse of any one of the preceding items, wherein the mouse comprises a differentiated human hematopoietic stem cell selected from the group consisting of a human myeloid progenitor cell, a human lymphoid progenitor cell, a human CD33⁺ myeloid cell, a human mast cell, a human monocyte, a human macrophage, human myeloid dendritic cell, a human B cell, a human plasma cell, a human T cell, a human T helper cell, a human cytotoxic T cell, a human T_{reg} cell, and a human natural killer cell.

Item 6. The genetically-modified, immunodeficient mouse of any one of the preceding items, wherein the mouse comprises a human leukocyte selected from the group consisting of CD45⁺, CD20⁺, CD20⁺CD45⁺, CD3⁺, CD3⁺CD45⁺, CD33⁺, CD33⁺CD45⁺, CD14⁺, CD14⁺CD45⁺, CD56⁺, and CD56⁺CD45⁺ leukocyte.

Item 7. The genetically-modified, immunodeficient mouse of item 6, wherein said mouse comprises, in the absence of an immunological challenge one, two, three or all of: at least about 20% of the human CD45⁺ leukocytes of the mouse are CD3⁺CD45⁺ leukocytes; at least about 10% of the human CD45⁺ leukocytes of the mouse are CD33⁺CD45⁺ leukocytes; at least about 5% of the human CD45⁺ leukocytes of the mouse are CD14⁺CD45⁺ leukocytes; at least about 0.5% of the human CD45⁺ leukocytes of the mouse are CD56⁺CD45⁺leukocytes.

Item 8. The genetically-modified, immunodeficient mouse of any one of items 4 to 7, wherein the mouse expresses a human cytokine selected from the group consisting of human interleukin-8, human interleukin-1β, human tumor-necrosis factor, human interleukin-12p70, and human interleukin-6.

Item 9. The genetically-modified, immunodeficient mouse of any one of the preceding items, further comprising a human xenograft comprising a human tumor cell.

Item 10. A method of making a genetically-modified, immunodeficient humanized mouse model, comprising: providing a genetically-modified, immunodeficient mouse according to any one of items 1 to 3; and administering human hematopoietic stem cells to the genetically-modified, immunodeficient mouse.

Item 11. The method of item 10, further comprising the step of: conditioning the genetically-modified, immunodeficient mouse to reduce mouse hematopoietic cells of the mouse prior to administering human hematopoietic stem cells.

Item 12. The method of item 11, wherein the conditioning step comprises irradiating the genetically-modified, immunodeficient mouse and/or administering a radiomimetic drug to the genetically-modified, immunodeficient mouse.

Item 13. The method of any one of items 10 to 12, further comprising the

step of: administering a human xenograft comprising a human tumor cell to the genetically-modified, immunodeficient mouse.

Item 14. The method of item 13, wherein the human hematopoietic stem cell and the human tumor cell comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 matching HLA alleles.

Item 15. A method of identifying anti-tumor activity of a test substance, comprising: providing a genetically-modified, immunodeficient mouse according to any one of items 1 to 8; administering a human tumor cell to the genetically-modified, immunodeficient mouse, wherein the human tumor cell form a solid or non-solid tumor in the genetically-modified, immunodeficient mouse; administering a test substance to the genetically-modified, immunodeficient mouse; and assaying a response of the solid or non-solid tumor to the test substance, wherein an inhibitory effect of the test substance on the tumor and/or tumor cells identifies the test substance as having anti-tumor activity

Item 16. The method of item 15, further comprising comparing the response to a standard to determine the effect of the test substance on the xenogeneic tumor cell, wherein an inhibitory effect of the test substance on the xenogeneic tumor cell identifies the test substance as having anti-tumor activity.

Item 17. The method of item 15 or 16, wherein the test substance is an

immunotherapeutic agent.

Item 18. The method of any one of items 15 to 17, wherein the test substance is an immune checkpoint inhibitor.

Item 19. The method of item 18, wherein the immune checkpoint inhibitor is a PD-1 inhibitor, PD-L1 inhibitor, or CTLA-4 inhibitor.

Item 20. The method of item 18 or 19, wherein the immune checkpoint inhibitor is atezolizumab, avelumab, durvalumab, ipilimumab, nivolumab, or pembrolizumab, or the immune checkpoint inhibitor comprises an antigen-binding fragment of any one of the foregoing.

Item 21. The method of any of items 15 to 20, wherein the test substance is an antibody.

Item 22. The method of any of items 15 to 21, wherein the test substance is an anti-cancer agent.

Item 23. A genetically-modified, immunodeficient mouse substantially as described or shown herein.

Item 24. A method of making a genetically-modified, immunodeficient humanized mouse model substantially as described or shown herein.

Item 25. A method of identifying anti-tumor activity of a test substance substantially as described or shown herein.

### Sequences

SEQ ID NO:1: Homo sapiens colony stimulating factor 1 (CSF1), transcript variant 1 encoding isoform a (based on NM_000757.5), 1662 nucleotides

ATGACCGCGCCGGGCGCCGCCGGGCGCTGCCCTCCCACGACATGGCTGGGCTCCCTGCT
GTTGTTGGTCTGTCTCCTGGCGAGCAGGAGTATCACCGAGGAGGTGTCGGAGTACTGTA
GCCACATGATTGGGAGTGGACACCTGCAGTCTCTGCAGCGGCTGATTGACAGTCAGATG
GAGACCTCGTGCCAAATTACATTTGAGTTTGTAGACCAGGAACAGTTGAAAGATCCAGT
GTGCTACCTTAAGAAGGCATTTCTCCTGGTACAAGACATAATGGAGGACACCATGCGCT
TCAGAGATAACACCCCCAATGCCATCGCCATTGTGCAGCTGCAGGAACTCTCTTTGAGG
CTGAAGAGCTGCTTCACCAAGGATTATGAAGAGCATGACAAGGCCTGCGTCCGAACTTT
CTATGAGACACCTCTCCAGTTGCTGGAGAAGGTCAAGAATGTCTTTAATGAAACAAAGA
ATCTCCTTGACAAGGACTGGAATATTTTCAGCAAGAACTGCAACAACAGCTTTGCTGAA
TGCTCCAGCCAAGATGTGGTGACCAAGCCTGATTGCAACTGCCTGTACCCCAAAGCCAT
CCCTAGCAGTGACCCGGCCTCTGTCTCCCCTCATCAGCCCCTCGCCCCCTCCATGGCCC
CTGTGGCTGGCTTGACCTGGGAGGACTCTGAGGGAACTGAGGGCAGCTCCCTCTTGCCT
GGTGAGCAGCCCCTGCACACAGTGGATCCAGGCAGTGCCAAGCAGCGGCCACCCAGGAG
CACCTGCCAGAGCTTTGAGCCGCCAGAGACCCCAGTTGTCAAGGACAGCACCATCGGTG
GCTCACCACAGCCTCGCCCCTCTGTCGGGGCCTTCAACCCCGGGATGGAGGATATTCTT
GACTCTGCAATGGGCACTAATTGGGTCCCAGAAGAAGCCTCTGGAGAGGCCAGTGAGAT
TCCCGTACCCCAAGGGACAGAGCTTTCCCCCTCCAGGCCAGGAGGGGGCAGCATGCAGA
CAGAGCCCGCCAGACCCAGCAACTTCCTCTCAGCATCTTCTCCACTCCCTGCATCAGCA
AAGGGCCAACAGCCGGCAGATGTAACTGGTACCGCCTTGCCCAGGGGGGCCCCGTGAG
GCCCACTGGCCAGGACTGGAATCACACCCCCCAGAAGACAGACCATCCATCTGCCCTGC
TCAGAGACCCCCCGGAGCCAGGCTCTCCCAGGATCTCATCACTGCGCCCCCAGGGCCTC
AGCAACCCCTCCACCCTCTCTGCTCAGCCACAGCTTTCCAGAAGCCACTCCTCGGGCAG
CGTGCTGCCCCTTGGGGAGCTGGAGGGCAGGAGGAGCACCAGGGATCGGAGGAGCCCCG
CAGAGCCAGAAGGAGGACCAGCAAGTGAAGGGGCAGCCAGGCCCCTGCCCCGTTTTAAC
TCCGTTCCTTTGACTGACACAGGCCATGAGAGGCAGTCCGAGGGATCCTCCAGCCCGCA
GCTCCAGGAGTCTGTCTTCCACCTGCTGGTGCCCAGTGTCATCCTGGTCTTGCTGGCCG
TCGGAGGCCTCTTGTTCTACAGGTGGAGGCGGCGGAGCCATCAAGAGCCTCAGAGAGCG
GATTCTCCCTTGGAGCAACCAGAGGGCAGCCCCCTGACTCAGGATGACAGACAGGTGGA
ACTGCCAGTG

SEQ ID NO:2: Homo sapiens colony stimulating factor 1 (CSF1), transcript variant 2 encoding isoform b (based on NM_172210.2), 1314 nucleotides

ATGACCGCGCCGGGCGCCGCCGGGCGCTGCCCTCCCACGACATGGCTGGGCTCCCTGCT
GTTGTTGGTCTGTCTCCTGGCGAGCAGGAGTATCACCGAGGAGGTGTCGGAGTACTGTA
GCCACATGATTGGGAGTGGACACCTGCAGTCTCTGCAGCGGCTGATTGACAGTCAGATG
GAGACCTCGTGCCAAATTACATTTGAGTTTGTAGACCAGGAACAGTTGAAAGATCCAGT
GTGCTACCTTAAGAAGGCATTTCTCCTGGTACAAGACATAATGGAGGACACCATGCGCT
TCAGAGATAACACCCCCAATGCCATCGCCATTGTGCAGCTGCAGGAACTCTCTTTGAGG
CTGAAGAGCTGCTTCACCAAGGATTATGAAGAGCATGACAAGGCCTGCGTCCGAACTTT
CTATGAGACACCTCTCCAGTTGCTGGAGAAGGTCAAGAATGTCTTTAATGAAACAAAGA
ATCTCCTTGACAAGGACTGGAATATTTTCAGCAAGAACTGCAACAACAGCTTTGCTGAA
TGCTCCAGCCAAGATGTGGTGACCAAGCCTGATTGCAACTGCCTGTACCCCAAAGCCAT
CCCTAGCAGTGACCCGGCCTCTGTCTCCCCTCATCAGCCCCTCGCCCCCTCCATGGCCC
CTGTGGCTGGCTTGACCTGGGAGGACTCTGAGGGAACTGAGGGCAGCTCCCTCTTGCCT
GGTGAGCAGCCCCTGCACACAGTGGATCCAGGCAGTGCCAAGCAGCGGCCACCCAGGAG
CACCTGCCAGAGCTTTGAGCCGCCAGAGACCCCAGTTGTCAAGGACAGCACCATCGGTG
GCTCACCACAGCCTCGCCCCTCTGTCGGGGCCTTCAACCCCGGGATGGAGGATATTCTT
GACTCTGCAATGGGCACTAATTGGGTCCCAGAAGAAGCCTCTGGAGAGGCCAGTGAGAT
TCCCGTACCCCAAGGGACAGAGCTTTCCCCCTCCAGGCCAGGAGGGGGCAGCATGCAGA
CAGAGCCCGCCAGACCCAGCAACTTCCTCTCAGCATCTTCTCCACTCCCTGCATCAGCA
AAGGGCCAACAGCCGGCAGATGTAACTGGCCATGAGAGGCAGTCCGAGGGATCCTCCAG
CCCGCAGCTCCAGGAGTCTGTCTTCCACCTGCTGGTGCCCAGTGTCATCCTGGTCTTGC
TGGCCGTCGGAGGCCTCTTGTTCTACAGGTGGAGGCGGCGGAGCCATCAAGAGCCTCAG
AGAGCGGATTCTCCCTTGGAGCAACCAGAGGGCAGCCCCCTGACTCAGGATGACAGACA
GGTGGAACTGCCAGTG

SEQ ID NO:3: Homo sapiens colony stimulating factor 1 (CSF1), transcript variant 3 encoding isoform c (based on NM_172211.3), 619 nucleotides

ATGACCGCGCCGGGCGCCGCCGGGCGCTGCCCTCCCACGACATGGCTGGGCTCCCTGCT
GTTGTTGGTCTGTCTCCTGGCGAGCAGGAGTATCACCGAGGAGGTGTCGGAGTACTGTA
GCCACATGATTGGGAGTGGACACCTGCAGTCTCTGCAGCGGCTGATTGACAGTCAGATG
GAGACCTCGTGCCAAATTACATTTGAGTTTGTAGACCAGGAACAGTTGAAAGATCCAGT
GTGCTACCTTAAGAAGGCATTTCTCCTGGTACAAGACATAATGGAGGACACCATGCGCT
TCAGAGATAACACCCCCAATGCCATCGCCATTGTGCAGCTGCAGGAACTCTCTTTGAGG
CTGAAGAGCTGCTTCACCAAGGATTATGAAGAGCATGACAAGGCCTGCGTCCGAACTTT
CTATGAGACACCTCTCCAGTTGCTGGAGAAGGTCAAGAATGTCTTTAATGAAACAAAGA
ATCTCCTTGACAAGGACTGGAATATTTTCAGCAAGAACTGCAACAACAGCTTTGCTGAA
TGCTCCAGCCAAGGCCATGAGAGGCAGTCCGAGGGATCCTCCAGCCCGCAGCTCCAGGA
GTCTGTCTTCCACCTGCTGGTGCCCAGTG

SEQ ID NO:4: Homo sapiens colony stimulating factor 1 (CSF1), transcript variant 4 encoding isoform a (based on NM_172212.2), 1662 nucleotides

ATGACCGCGCCGGGCGCCGCCGGGCGCTGCCCTCCCACGACATGGCTGGGCTCCCTGCT
GTTGTTGGTCTGTCTCCTGGCGAGCAGGAGTATCACCGAGGAGGTGTCGGAGTACTGTA
GCCACATGATTGGGAGTGGACACCTGCAGTCTCTGCAGCGGCTGATTGACAGTCAGATG
GAGACCTCGTGCCAAATTACATTTGAGTTTGTAGACCAGGAACAGTTGAAAGATCCAGT
GTGCTACCTTAAGAAGGCATTTCTCCTGGTACAAGACATAATGGAGGACACCATGCGCT
TCAGAGATAACACCCCCAATGCCATCGCCATTGTGCAGCTGCAGGAACTCTCTTTGAGG
CTGAAGAGCTGCTTCACCAAGGATTATGAAGAGCATGACAAGGCCTGCGTCCGAACTTT
CTATGAGACACCTCTCCAGTTGCTGGAGAAGGTCAAGAATGTCTTTAATGAAACAAAGA
ATCTCCTTGACAAGGACTGGAATATTTTCAGCAAGAACTGCAACAACAGCTTTGCTGAA
TGCTCCAGCCAAGATGTGGTGACCAAGCCTGATTGCAACTGCCTGTACCCCAAAGCCAT
CCCTAGCAGTGACCCGGCCTCTGTCTCCCCTCATCAGCCCCTCGCCCCCTCCATGGCCC
CTGTGGCTGGCTTGACCTGGGAGGACTCTGAGGGAACTGAGGGCAGCTCCCTCTTGCCT
GGTGAGCAGCCCCTGCACACAGTGGATCCAGGCAGTGCCAAGCAGCGGCCACCCAGGAG
CACCTGCCAGAGCTTTGAGCCGCCAGAGACCCCAGTTGTCAAGGACAGCACCATCGGTG
GCTCACCACAGCCTCGCCCCTCTGTCGGGGCCTTCAACCCCGGGATGGAGGATATTCTT
GACTCTGCAATGGGCACTAATTGGGTCCCAGAAGAAGCCTCTGGAGAGGCCAGTGAGAT
TCCCGTACCCCAAGGGACAGAGCTTTCCCCCTCCAGGCCAGGAGGGGGCAGCATGCAGA
CAGAGCCCGCCAGACCCAGCAACTTCCTCTCAGCATCTTCTCCACTCCCTGCATCAGCA
AAGGGCCAACAGCCGGCAGATGTAACTGGTACCGCCTTGCCCAGGGTGGGCCCCGTGAG
GCCCACTGGCCAGGACTGGAATCACACCCCCCAGAAGACAGACCATCCATCTGCCCTGC
TCAGAGACCCCCCGGAGCCAGGCTCTCCCAGGATCTCATCACTGCGCCCCCAGGGCCTC
AGCAACCCCTCCACCCTCTCTGCTCAGCCACAGCTTTCCAGAAGCCACTCCTCGGGCAG
CGTGCTGCCCCTTGGGGAGCTGGAGGGCAGGAGGAGCACCAGGGATCGGAGGAGCCCCG
CAGAGCCAGAAGGAGGACCAGCAAGTGAAGGGGCAGCCAGGCCCCTGCCCCGTTTTAAC
TCCGTTCCTTTGACTGACACAGGCCATGAGAGGCAGTCCGAGGGATCCTCCAGCCCGCA
GCTCCAGGAGTCTGTCTTCCACCTGCTGGTGCCCAGTGTCATCCTGGTCTTGCTGGCCG
TCGGAGGCCTCTTGTTCTACAGGTGGAGGCGGCGGAGCCATCAAGAGCCTCAGAGAGCG
GATTCTCCCTTGGAGCAACCAGAGGGCAGCCCCCTGACTCAGGATGACAGACAGGTGGA
ACTGCCAGTG

SEQ ID NO:5: Amino acid sequence of human colony-stimulating factor 1 (hCSF1) isoform a—554 amino acids (including 32 amino acid N-terminal signal peptide) (NP_000748.3)

MTAPGAAGRCPPTTWLGSLLLLVCLLASRSITEEVSEYCSHMIGSGHLQSLQRLIDSQM
ETSCQITFEFVDQEQLKDPVCYLKKAFLLVQDIMEDTMRFRDNTPNAIAIVQLQELSLR
LKSCFTKDYEEHDKACVRTFYETPLQLLEKVKNVFNETKNLLDKDWNIFSKNCNNSFAE
CSSQDVVTKPDCNCLYPKAIPSSDPASVSPHQPLAPSMAPVAGLTWEDSEGTEGSSLLP
GEQPLHTVDPGSAKQRPPRSTCQSFEPPETPVVKDSTIGGSPQPRPSVGAFNPGMEDIL
DSAMGTNWVPEEASGEASEIPVPQGTELSPSRPGGGSMQTEPARPSNFLSASSPLPASA
KGQQPADVTGTALPRVGPVRPTGQDWNHTPQKTDHPSALLRDPPEPGSPRISSLRPQGL
SNPSTLSAQPQLSRSHSSGSVLPLGELEGRRSTRDRRSPAEPEGGPASEGAARPLPRFN
SVPLTDTGHERQSEGSSSPQLQESVFHLLVPSVILVLLAVGGLLFYRWRRRSHQEPQRA
DSPLEQPEGSPLTQDDRQVELPV

SEQ ID NO:6: Amino acid sequence of human colony-stimulating factor 1 (hCSF1) without signal peptide—522 amino acids (based on NP_000748.3)

EEVSEYCSHMIGSGHLQSLQRLIDSQMETSCQITFEFVDQEQLKDPVCYLKKAFLLVQD
IMEDTMRFRDNTPNAIAIVQLQELSLRLKSCFTKDYEEHDKACVRTFYETPLQLLEKVK
NVFNETKNLLDKDWNIFSKNCNNSFAECSSQDVVTKPDCNCLYPKAIPSSDPASVSPHQ
PLAPSMAPVAGLTWEDSEGTEGSSLLPGEQPLHTVDPGSAKQRPPRSTCQSFEPPETPV
VKDSTIGGSPQPRPSVGAFNPGMEDILDSAMGINWVPEEASGEASEIPVPQGTELSPSR
PGGGSMQTEPARPSNFLSASSPLPASAKGQQPADVTGTALPRVGPVRPTGQDWNHTPQK
TDHPSALLRDPPEPGSPRISSLRPQGLSNPSTLSAQPQLSRSHSSGSVLPLGELEGRRS
TRDRRSPAEPEGGPASEGAARPLPRFNSVPLTDTGHERQSEGSSSPQLQESVFHLLVPS
VILVLLAVGGLLFYRWRRRSHQEPQRADSPLEQPEGSPLTQDDRQVELPV

SEQ ID NO:7: Amino acid sequence of human colony-stimulating factor 1 (hCSF1) isoform b—438 amino acids (including 32 amino acid N-terminal signal peptide)—(NP_757349.1) encoded by transcript variant 2

MTAPGAAGRCPPTTWLGSLLLLVCLLASRSITEEVSEYCSHMIGSGHLQSLQRLIDSQM
ETSCQITFEFVDQEQLKDPVCYLKKAFLLVQDIMEDTMRFRDNTPNAIAIVQLQELSLR
LKSCFTKDYEEHDKACVRTFYETPLQLLEKVKNVFNETKNLLDKDWNIFSKNCNNSFAE
CSSQDVVTKPDCNCLYPKAIPSSDPASVSPHQPLAPSMAPVAGLTWEDSEGTEGSSLLP
GEQPLHTVDPGSAKQRPPRSTCQSFEPPETPVVKDSTIGGSPQPRPSVGAFNPGMEDIL
DSAMGTNWVPEEASGEASEIPVPQGTELSPSRPGGGSMQTEPARPSNFLSASSPLPASA
KGQQPADVTGHERQSEGSSSPQLQESVFHLLVPSVILVLLAVGGLLFYRWRRRSHQEPQ
RADSPLEQPEGSPLTQDDRQVELPV

SEQ ID NO:8: Amino acid sequence of human colony-stimulating factor 1 (hCSF1) isoform b without signal peptide—406 amino acids (based on NP_757349.1)

EEVSEYCSHMIGSGHLQSLQRLIDSQMETSCQITFEFVDQEQLKDPVCYLKKAFLLVQD
IMEDTMRFRDNTPNAIAIVQLQELSLRLKSCFTKDYEEHDKACVRTFYETPLQLLEKVK
NVFNETKNLLDKDWNIFSKNCNNSFAECSSQDVVTKPDCNCLYPKAIPSSDPASVSPHQ
PLAPSMAPVAGLTWEDSEGTEGSSLLPGEQPLHTVDPGSAKQRPPRSTCQSFEPPETPV
VKDSTIGGSPQPRPSVGAFNPGMEDILDSAMGTNWVPEEASGEASEIPVPQGTELSPSR
PGGGSMQTEPARPSNFLSASSPLPASAKGQQPADVTGHERQSEGSSSPQLQESVFHLLV
PSVILVLLAVGGLLFYRWRRRSHQEPQRADSPLEQPEGSPLTQDDRQVELPV

SEQ ID NO:9: Amino acid sequence of human colony-stimulating factor 1 (hCSF1) isoform c—256 amino acids—(including 32 amino acid N-terminal signal peptide) (NP_757350.1) encoded by transcript variant 3

MTAPGAAGRCPPTTWLGSLLLLVCLLASRSITEEVSEYCSHMIGSGHLQSLQRLIDSQM
ETSCQITFEFVDQEQLKDPVCYLKKAFLLVQDIMEDTMRFRDNTPNAIAIVQLQELSLR
LKSCFTKDYEEHDKACVRTFYETPLQLLEKVKNVFNETKNLLDKDWNIFSKNCNNSFAE
CSSQGHERQSEGSSSPQLQESVFHLLVPSVILVLLAVGGLLFYRWRRRSHQEPQRADSP
LEQPEGSPLTQDDRQVELPV

SEQ ID NO:10: Amino acid sequence of human colony-stimulating factor 1 (hCSF1) isoform c without signal peptide—224 amino acids (based on NP_757350.1)

EEVSEYCSHMIGSGHLQSLQRLIDSQMETSCQITFEFVDQEQLKDPVCYLKKAFLLVQD
IMEDTMRFRDNTPNAIAIVQLQELSLRLKSCFTKDYEEHDKACVRTFYETPLQLLEKVK
NVFNETKNLLDKDWNIFSKNCNNSFAECSSQGHERQSEGSSSPQLQESVFHLLVPSVIL
VLLAVGGLLFYRWRRRSHQEPQRADSPLEQPEGSPLTQDDRQVELPV

SEQ ID NO. 11: human SCF 220 (245 aa)

MKKTQTWILTCIYLQLLLFNPLVKTEGICRNRVTNNVKDVTKLVANLPKD
YMITLKYVPGMDVLPSHCWISEMVVQLSDSLTDLLDKFSNISEGLSNYSI
IDKLVNIVDDLVECVKENSSKDLKKSFKSPEPRLFTPEEFFRIFNRSIDA
FKDFVVASETSDCVVSSTLSPEKGKAKNPPGDSSLHWAAMALPALFSLII
GFAFGALYWKKRQPSLTRAVENIQINEEDNEISMLQEKEREFQEV

SEQ ID NO. 12: Nucleotide Sequence encoding human SCF 220, 738 nucleotides

ATGAAGAAGACACAAACTTGGATTCTCACTTGCATTTATCTTCAGCTGCT
CCTATTTAATCCTCTCGTCAAAACTGAAGGGATCTGCAGGAATCGTGTGA
CTAATAATGTAAAAGACGTCACTAAATTGGTGGCAAATCTTCCAAAAGAC
TACATGATAACCCTCAAATATGTCCCCGGGATGGATGTTTTGCCAAGTCA
TTGTTGGATAAGCGAGATGGTAGTACAATTGTCAGACAGCTTGACTGATC
TTCTGGACAAGTTTTCAAATATTTCTGAAGGCTTGAGTAATTATTCCATC
ATAGACAAACTTGTGAATATAGTGGATGACCTTGTGGAGTGCGTGAAAGA
AAACTCATCTAAGGATCTAAAAAAATCATTCAAGAGCCCAGAACCCAGGC
TCTTTACTCCTGAAGAATTCTTTAGAATTTTTAATAGATCCATTGATGCC
TTCAAGGACTTTGTAGTGGCATCTGAAACTAGTGATTGTGTGGTTTCTTC
AACATTAAGTCCTGAGAAAGGGAAGGCCAAAAATCCCCCTGGAGACTCCA
GCCTACACTGGGCAGCCATGGCATTGCCAGCATTGTTTTCTCTTATAATT
GGCTTTGCTTTTGGAGCCTTATACTGGAAGAAGAGACAGCCAAGTCTTAC
AAGGGCAGTTGAAAATATACAAATTAATGAAGAGGATAATGAGATAAGTA
TGTTGCAAGAGAAAGAGAGAGAGTTTCAAGAAGTGTAA

SEQ ID NO. 13: human SCF 248 (273 aa)

MKKTQTWILTCIYLQLLLFNPLVKTEGICRNRVTNNVKDVTKLVANLPKD
YMITLKYVPGMDVLPSHCWISEMVVQLSDSLTDLLDKFSNISEGLSNYSI
IDKLVNIVDDLVECVKENSSKDLKKSFKSPEPRLFTPEEFFRIFNRSIDA
FKDFVVASETSDCVVSSTLSPEKDSRVSVTKPFMLPPVAASSLRNDSSSS
NRKAKNPPGDSSLHWAAMALPALFSLIIGFAFGALYWKKROPSLTRAVEN
IQINEEDNEISMLQEKEREFQEV

SEQ ID NO. 14: Nucleotide Sequence encoding human SCF 248. 822 nucleotides

ATGAAGAAGACACAAACTTGGATTCTCACTTGCATTTATCTTCAGCTGCT
CCTATTTAATCCTCTCGTCAAAACTGAAGGGATCTGCAGGAATCGTGTGA
CTAATAATGTAAAAGACGTCACTAAATTGGTGGCAAATCTTCCAAAAGAC
TACATGATAACCCTCAAATATGTCCCCGGGATGGATGTTTTGCCAAGTCA
TTGTTGGATAAGCGAGATGGTAGTACAATTGTCAGACAGCTTGACTGATC
TTCTGGACAAGTTTTCAAATATTTCTGAAGGCTTGAGTAATTATTCCATC
ATAGACAAACTTGTGAATATAGTGGATGACCTTGTGGAGTGCGTGAAAGA
AAACTCATCTAAGGATCTAAAAAAATCATTCAAGAGCCCAGAACCCAGGC
TCTTTACTCCTGAAGAATTCTTTAGAATTTTTAATAGATCCATTGATGCC
TTCAAGGACTTTGTAGTGGCATCTGAAACTAGTGATTGTGTGGTTTCTTC
AACATTAAGTCCTGAGAAAGATTCCAGAGTCAGTGTCACAAAACCATTTA
TGTTACCCCCTGTTGCAGCCAGCTCCCTTAGGAATGACAGCAGTAGCAGT
AATAGGAAGGCCAAAAATCCCCCTGGAGACTCCAGCCTACACTGGGCAGC
CATGGCATTGCCAGCATTGTTTTCTCTTATAATTGGCTTTGCTTTTGGAG
CCTTATACTGGAAGAAGAGACAGCCAAGTCTTACAAGGGCAGTTGAAAAT
ATACAAATTAATGAAGAGGATAATGAGATAAGTATGTTGCAAGAGAAAGA
GAGAGAGTTTCAAGAAGTGTAA

SEQ ID NO.15: nucleotide sequence encoding human SCF 248 including 5′ and 3′ non-coding sequences, 1405 nucleotides

CCGCCTCGCGCCGAGACTAGAAGCGCTGCGGGAAGCAGGGACAGTGGAGA
GGGCGCTGCGCTCGGGCTACCCAATGCGTGGACTATCTGCCGCCGCTGTT
CGTGCAATATGCTGGAGCTCCAGAACAGCTAAACGGAGTCGCCACACCAC
TGTTTGTGCTGGATCGCAGCGCTGCCTTTCCTTATGAAGAAGACACAAAC
TTGGATTCTCACTTGCATTTATCTTCAGCTGCTCCTATTTAATCCTCTCG
TCAAAACTGAAGGGATCTGCAGGAATCGTGTGACTAATAATGTAAAAGAC
GTCACTAAATTGGTGGCAAATCTTCCAAAAGACTACATGATAACCCTCAA
ATATGTCCCCGGGATGGATGTTTTGCCAAGTCATTGTTGGATAAGCGAGA
TGGTAGTACAATTGTCAGACAGCTTGACTGATCTTCTGGACAAGTTTTCA
AATATTTCTGAAGGCTTGAGTAATTATTCCATCATAGACAAACTTGTGAA
TATAGTGGATGACCTTGTGGAGTGCGTGAAAGAAAACTCATCTAAGGATC
TAAAAAAATCATTCAAGAGCCCAGAACCCAGGCTCTTTACTCCTGAAGAA
TTCTTTAGAATTTTTAATAGATCCATTGATGCCTTCAAGGACTTTGTAGT
GGCATCTGAAACTAGTGATTGTGTGGTTTCTTCAACATTAAGTCCTGAGA
AAGATTCCAGAGTCAGTGTCACAAAACCATTTATGTTACCCCCTGTTGCA
GCCAGCTCCCTTAGGAATGACAGCAGTAGCAGTAATAGGAAGGCCAAAAA
TCCCCCTGGAGACTCCAGCCTACACTGGGCAGCCATGGCATTGCCAGCAT
TGTTTTCTCTTATAATTGGCTTTGCTTTTGGAGCCTTATACTGGAAGAAG
AGACAGCCAAGTCTTACAAGGGCAGTTGAAAATATACAAATTAATGAAGA
GGATAATGAGATAAGTATGTTGCAAGAGAAAGAGAGAGAGTTTCAAGAAG
TGTAAATTGTGGCTTGTATCAACACTGTTACTTTCGTACATTGGCTGGTA
ACAGTTCATGTTTGCTTCATAAATGAAGCAGCTTTAAACAAATTCATATT
CTGTCTGGAGTGACAGACCACATCTTTATCTGTTCTTGCTACCCATGACT
TTATATGGATGATTCAGAAATTGGAACAGAATGTTTTACTGTGAAACTGG
CACTGAATTAATCATCTATAAAGAAGAACTTGCATGGAGCAGGACTCTAT
TTTAAGGACTGCGGGACTTGGGTCTCATTTAGAACTTGCAGCTGATGTTG
GAAGAGAAAGCACGTGTCTCAGACTGCATGTACCATTTGCATGGCTCCAG
AAATGTCTAAATGCTGAAAAAACACCTAGCTTTATTCTTCAGATACAAAC
TGCAG

SEQ ID NO. 16: human soluble SCF (164 aa and 25 aa N-terminal signal peptide)

MKKTQTWILTCIYLQLLLFNPLVKTEGICRNRVINNVKDVTKLVANLPKD
YMITLKYVPGMDVLPSHCWISEMVVQLSDSLTDLLDKFSNISEGLSNYSI
IDKLVNIVDDLVECVKENSSKDLKKSFKSPEPRLFTPEEFFRIFNRSIDA
FKDFVVASETSDCVVSSTLSPEKDSRVSVTKPFMLPPVA

SEQ ID NO. 17: nucleotide sequence encoding human soluble SCF (164 aa and 25 aa N-terminal signal peptide), 567 nucleotides

ATGAAGAAGACACAAACTTGGATTCTCACTTGCATTTATCTTCAGCTGCT
CCTATTTAATCCTCTCGTCAAAACTGAAGGGATCTGCAGGAATCGTGTGA
CTAATAATGTAAAAGACGTCACTAAATTGGTGGCAAATCTTCCAAAAGAC
TACATGATAACCCTCAAATATGTCCCCGGGATGGATGTTTTGCCAAGTCA
TTGTTGGATAAGCGAGATGGTAGTACAATTGTCAGACAGCTTGACTGATC
TTCTGGACAAGTTTTCAAATATTTCTGAAGGCTTGAGTAATTATTCCATC
ATAGACAAACTTGTGAATATAGTGGATGACCTTGTGGAGTGCGTGAAAGA
AAACTCATCTAAGGATCTAAAAAAATCATTCAAGAGCCCAGAACCCAGGC
TCTTTACTCCTGAAGAATTCTTTAGAATTTTTAATAGATCCATTGATGCC
TTCAAGGACTTTGTAGTGGCATCTGAAACTAGTGATTGTGTGGTTTCTTC
AACATTAAGTCCTGAGAAAGATTCCAGAGTCAGTGTCACAAAACCATTTA
TGTTACCCCCTGTTGCA

SEQ ID NO:18: hGM-CSF—144 amino acids (including 17 amino acid signal peptide)

MWLQSLLLLGTVACSISAPARSPSPSTQPWEHVNAIQEARRLLNLSRDTA
AEMNETVEVISEMFDLQEPTCLQTRLELYKOGLRGSLTKLKGPLTMMASH
YKQHCPPTPETSCATQIITFESFKENLKDFLLVIPFDCWEPVQE

SEQ ID NO:19: Nucleotide sequence encoding hGM-CSF (including 17 amino acid signal peptide)—435 nucleotides

atgtggctgcagagcctgctgctcttgggcactgtggcctgcagcatctc
tgcacccgcccgctcgcccagccccagcacgcagccctgggagcatgtga
atgccatccaggaggcccggcgtctcctgaacctgagtagagacactgct
gctgagatgaatgaaacagtagaagtcatctcagaaatgtttgacctcca
ggagccgacctgcctacagacccgcctggagctgtacaagcagggcctgc
ggggcagcctcaccaagctcaagggccccttgaccatgatggccagccac
tacaagcagcactgccctccaaccccggaaacttcctgtgcaacccagat
tatcacctttgaaagtttcaaagagaacctgaaggactttctgcttgtca
tcccctttgactgctgggagccagtccaggagtga

SEQ ID NO:20: hGM-CSF without signal peptide—127 amino acids

APARSPSPSTOPWEHVNAIQEARRLLNLSRDTAAEMNETVEVISEMFDLQ
EPTCLQTRLELYKQGLRGSLTKLKGPLTMMASHYKQHCPPTPETSCATQI
ITFESFKENLKDELLVIPFDCWEPVQE

SEQ ID NO:21: Nucleotide sequence encoding hGM-CSF protein without signal peptide—352 nucleotides

gccctgggagcatgtgaatgccatccaggaggcccggcgtctcctgaacc
tgagtagagacactgctgctgagatgaatgaaacagtagaagtcatctca
gaaatgtttgacctccaggagccgacctgcctacagacccgcctggagct
gtacaagcagggcctgcggggcagcctcaccaagctcaagggccccttga
ccatgatggccagccactacaagcagcactgccctccaaccccggaaact
tcctgtgcaacccagattatcacctttgaaagtttcaaagagaacctgaa
ggactttctgcttgtcatcccctttgactgctgggagccagtccaggagt
ga

SEQ ID NO: 22: human interleukin-3 152 amino acids (including 19 amino acid signal peptide)

MSRLPVLLLLQLLVRPGLQAPMTQTTPLKTSWVNCSNMIDEIITHLKQPP
LPLLDENNLNGEDQDILMENNLRRPNLEAFNRAVKSLONASAIESILKNL
LPCLPLATAAPTRHPIHIKDGDWNEFRRKLTFYLKTLENAQAQQTTLSLA
IF

SEQ ID NO: 23: Nucleotide sequence encoding interleukin-3 (including 19 amino acid signal peptide)—459 nucleotides

atgagccgcctgcccgtcctgctcctgctccaactcctggtccgccccgg
actccaagctcccatgacccagacaacgcccttgaagacaagctgggtta
actgctctaacatgatcgatgaaattataacacacttaaagcagccacct
ttgcctttgctggacttcaacaacctcaatggggaagaccaagacattct
gatggaaaataaccttcgaaggccaaacctggaggcattcaacagggctg
tcaagagtttacagaacgcatcagcaattgagagcattcttaaaaatctc
ctgccatgtctgcccctggccacggccgcacccacgcgacatccaatcca
tatcaaggacggtgactggaatgaattccggaggaaactgacgttctatc
tgaaaacccttgagaatgcgcaggctcaacagacgactttgagcctcgcg
atcttttga

SEQ ID NO: 24: human interleukin-3 without signal peptide—133 amino acids

APMTQTTPLKTSWVNCSNMIDEIITHLKQPPLPLLDENNLNGEDQDILME
NNLRRPNLEAFNRAVKSLQNASAIESILKNLLPCLPLATAAPTRHPIHIK
DGDWNEFRRKLTFYLKTLENAQAQQTTLSLAIF

SEQ ID NO: 25: Nucleotide sequence encoding interleukin-3 without signal peptide—402 nucleotides

gctcccatgacccagacaacgcccttgaagacaagctgggttaactgctc
taacatgatcgatgaaattataacacacttaaagcagccacctttgcctt
tgctggacttcaacaacctcaatggggaagaccaagacattctgatggaa
aataaccttcgaaggccaaacctggaggcattcaacagggctgtcaagag
tttacagaacgcatcagcaattgagagcattcttaaaaatctcctgccat
gtctgcccctggccacggccgcacccacgcgacatccaatccatatcaag
gacggtgactggaatgaattccggaggaaactgacgttctatctgaaaac
ccttgagaatgcgcaggctcaacagacgactttgagcctcgcgatctttt
ga

1.-12. (canceled)

13. An immunodeficient transgenic mouse whose genome expresses a transgene encoding human stem cell factor (hSCF), a transgene encoding human granulocyte-macrophage colony-stimulating factor (hGM-CSF), a transgene encoding human interleukin-3 (hIL-3), and a transgene encoding human colony-stimulating factor 1 (hCSF1).

14. The immunodeficient transgenic mouse of claim 13, wherein the genome of the immunodeficient transgenic mouse is homozygous for a Prkdc^{scid} allele.

15. The immunodeficient transgenic mouse of claim 13, wherein the genome of the immunodeficient transgenic mouse is homozygous for a Il2rg^{tm1Wjl} allele.

16. The immunodeficient transgenic mouse of claim 13, wherein the genome of the immunodeficient transgenic mouse is homozygous for the Prkdc^{scid} allele and is homozygous for the Il2rg^{tm1Wjl} allele.

17. The immunodeficient transgenic mouse of claim 13, wherein the immunodeficient transgenic mouse has been humanized, wherein the immunodeficient transgenic mouse:
(a) comprises fewer numbers of human CD20+ cells and increased numbers of human CD3+ cells, CD33+ cells, CD14+ cells, and CD56+ cells as a proportion of human CD45+ cells relative to a control mouse, and
(b) displays an increase in the relative number of human CD14+ macrophages and human CD56+ natural killer cells and increased concentrations of macrophage-secreted cytokines relative to a control mouse, and
wherein the control mouse is an immunodeficient transgenic mouse whose genome expresses a transgene encoding hSCF, a transgene encoding hGM-CSF, and a transgene encoding hIL-3, and not a transgene encoding hCSF1.

18. The immunodeficient transgenic mouse of claim 17, wherein the immunodeficient transgenic mouse has been engrafted with a human tumor xenograft.

19. The immunodeficient transgenic mouse of claim 17, wherein in the absence of an immunological challenge peripheral blood of the immunodeficient transgenic mouse comprises human CD45+ cells, and wherein:
at least about 20% of the human CD45+ cells are CD3+CD45+ cells;
at least about 10% of the human CD45+ cells are CD33+CD45+ cells;
at least about 5% of the human CD45+ cells are CD14+CD45+ cells; and/or
at least about 0.5% of the human CD45+ cells are CD56+CD45+ cells.

20. The immunodeficient transgenic mouse of claim 17, wherein serum of the immunodeficient transgenic mouse comprises human cytokines.

21. An immunodeficient transgenic mouse whose genome:
(a) expresses a transgene encoding human stem cell factor (hSCF), a transgene encoding human granulocyte-macrophage colony-stimulating factor (hGM-CSF), a transgene encoding human interleukin-3 (hIL-3), and a transgene encoding human colony-stimulating factor 1 (hCSF1); and
(b) is homozygous for a Prkdc^{scid} allele and is homozygous for a Il2rg^{tm1Wjl} allele,
wherein the immunodeficient transgenic mouse has been humanized and engrafted with a human tumor xenograft.

22. The immunodeficient transgenic mouse of claim 21, wherein serum of the immunodeficient transgenic mouse comprises human cytokines.
